(19) United States
(12) Patent Application Publication
(10) Pub. No.: US 2004/0210047 A1
(43) Pub. Date: Oct. 21, 2004

(54) Methallyl sucroses and their epoxy derivatives

(76) Inventors: Navzer D. Sachinvala, New Orleans, LA (US); Morton H. Litt, Cleveland, OH (US); David Winsor, Hickory, NC (US)

Correspondence Address: G. BYRON STOVER, USDA, ABS OTT, 5601 SUNNYSIDE AVE., RM 4-1159, BELTSVILLE, MD, 20705-5131, US

(21) Appl. No.: 10/417,067

(22) Filed: Apr. 15, 2003

(51) Int. Cl. C07H 15/04

(52) U.S. Cl. 536/120

(57) ABSTRACT

Saccharide-based epoxy resins such as epoxies, adhesives, coatings and composites, made from methods using unepoxidized saccharide-based monomers, and saccharide-based epoxidized monomers and polymers, especially derived from sucrose.

### (D) MICROFICHE APPENDIX

Not applicable.

### (E) BACKGROUND OF THE INVENTION

(1) Field of the Invention

The subject invention relates to saccharide-based ether monomers containing double bonds and their epoxy monomers suitable for the production of polymeric saccharide-based epoxy resins or thermosets, together with methods for producing such monomers and polymer resins. The invention also generally relates to novel sucrose derivatives useful for preparing epoxy monomers and their polymerizable mixtures, especially those capable of curing relatively quickly and/or at relatively low curing temperatures, and/or resulting in thermosets.

(2) Description of the Related Art Including Information Disclosed Under 37 C.F.R. 1.97 and 1.98.

U.S. Pat. No. 5,571,907 issued to two of the inventors listed herein discloses a method for producing sucrose-based epoxy monomers having one to eight epoxy groups per molecule of sucrose, comprising reacting a mixture comprising a sucrose monomer having an allyl-containing group on at least one of the hydroxyl groups, and a reagent or catalyst, to produce an epoxy monomer. Said monomers can then be cured to produce epoxy resins useful for adhesives, composites and coatings.

U.S. Pat. No. 6,646,226 issued to two of the inventors listed herein discloses a method for producing a crosslinked saccharide-based resin (together with the resin produced by said method), and a method for preparing saccharide-based polymers; said patent also discloses an adhesive, coating and reinforced material, each comprising a sucrose-based resin.

An epoxide, or oxirane, is a three membered ring (cyclic ether) containing two adjacent methylenes or methines and an oxygen. Epoxides are derivatives of ethylene oxide. Compounds containing epoxide groups are important because epoxides are highly reactive moieties that are useful starting materials for the synthetic chemist. In particular, epoxides are capable of being attacked by both electrophiles and nucleophiles. Epoxide chemistry is widely described in the literature such as in polymer chemistry texts and reviews (see, for example, Tanaka, Y. in Epoxy Resins: Chemistry and Technology, 2^{nd} Edition, May, C. A. (editor), Marcel Dekker, New York, 1988, pp 9-284; Odian, G. Principles of Polymerization, 3^{rd} Edition, J. Wiley & Sons, New York, 1991, pp 134-136; Stevens, M. P. Polymer Chemistry, 2^{nd} Edition, Oxford University Press, New York, 1990, pp. 329-351; and Bauer, R. S. in Epoxy Resins; Chemistry and Technology, A.C.S. Audio Course, American Chemical Society, Washington, D.C., 1991).

Commercial epoxy resins are oligomeric materials that contain one or more epoxy or oxirane group per molecule. The most widely used epoxy resins are the diglycidyl ethers of bisphenol-A obtained upon reaction of bisphenol-A with epichlorohydrin (see, May, C. A. in Epoxy Resins: Chemistry and Technology, 2^{nd} Edition, May, C. A. (editor), Marcel Dekker, 2^{nd} Edition, New York, 988, pp 1-8).

Since their introduction in the late nineteen forties, epoxy resins have permeated many technologies. They are used extensively in adhesives, reinforced materials, and as coatings. As adhesives, epoxy resins are used to bind concrete, glass, wood, metals, and plastic surfaces. As coatings, because of their chemical resistance and excellent corrosion protection, they are used as primers in the maintenance of on and off shore refineries, drums, pails, and food and beverage containers since they are chemically inert, non-toxic and impart no taste when fully cured. In structural applications, epoxy resins find use in potting and encapsulation of electrical equipment; adhesives for automobile and aircraft manufacturing; sealants in flooring and paving applications; grouting agents; and reinforced composites for the construction of pipes, tanks, aircraft and automobile components. These structural applications are possible because the related epoxy resins set quickly enough and have solvent and chemical resistance; they also exhibit low shrinkage upon cure, as well as excellent electrical, thermal and moisture resistance. However, many epoxy resins have some special storage and handling requirements (see, May, C. A. in Epoxy Resins: Chemistry and Technology, 2^{nd} Edition, May, C. A. (editor), Marcel Dekker, 2^{nd} Edition, New York, 1988, pp 1-8).

There exists a need in the art for improved epoxy monomers which can be used for preparing epoxy polymers and resins that curing more efficiently. Moreover, given the various uses for epoxy polymers and resins, it is clear that improved epoxide monomers and polymers with better physical properties resulting from these methods are highly desirable.

### (F) BRIEF SUMMARY OF THE INVENTION

In most general form, the invention disclosed herein includes a method of making saccharide monomers with olefins (double bonds) using a saccharide such as sucrose. The invention also includes a method of epoxidizing said monomers to form epoxy monomers. The present invention also includes a method of polymerizing said epoxy monomers into thermosets.

Sucrose was reacted in aqueous sodium hydroxide with inethallyl chloride in a Parr reactor to form a mixture of methallyl sucroses (“MS”, >90% yields), by modified methods of Nichols, P. L and Yanovski, E., J. Am. Chem. Soc., 1944, 66, 1625. In addition, sucrose was also transformed to octa-O-methallylsucrose by the method of Sachinvala (J. Polymer Science. Polymer Chemistry Ed., 1995, 33, 15-29) using sodium hydride, a suitable polar aprotic solvent such as dimethylsulfoxide (“DMSO”), dimethylformamide (“DMF”) or dimethylacetamide (“DMAc”), and methallyl chloride. The methallyl sucrose ethers were then epoxidized with peracids to generate epoxymethallyl sucroses (“EMS”) in 95% yield (by method similar to that of Sachinvala, et al., U.S. Pat. No. 5,571,907 and U.S. Pat. No. 5,646,226. MS produced from the aqueous preparation was found to contain an average of ˜7 methallyl ether groups, and MS prepared in aprotic solvents usually contained 8 methallyl ethers. Changing the amount of methallyl chloride added to the aqueous preparation changes the amount of methallyl groups from 1 to about 7 and, changing the amount of methallyl chloride added to the aprotic solvent preparations changes the amount of methallyl from 1 to about 8. The mixture of EMS, using MS from the aqueous preparation, was found to contain an average of ˜7 methallyl groups per sucrose, and ˜5 epoxy groups per sucrose. And EMS using MS from the aprotic solvent preparation contained 8 methallyl groups, and ˜6 epoxy groups.

EMS monomers are analogs of the previously discovered epoxyallyl and epoxycrotyl sucroses (“EAS” and “ECS”, respectively). EMS contains geminally disubstituted terminal double bonds and geminally disubstituted terminal epoxy groups. Surprisingly, it behaves differently than EAS and ECS; it is also more reactive than EAS and ECS, and the commercial epoxy diglycidyl ether of bisphenol-A (“DGEBA”).

EMS was found to cure about 10° C. to 20° C. below any known commercial epoxy with nucleophiles and electrophiles. Furthermore, EMS, unlike EAS and ECS, readily cured with itself, partially epoxidized EAS and ECS, and with allyl sucrose (AS), crotyl sucrose (CS), and MS when gently heated in the presence of electrophilic catalysts. The cured EMS thermosets are clear and tough, and tenaciously bind dissimilar surfaces regardless of geometry. According to the Maron-Ames tests (Maron, D., Ames, B. Mutation Res., 1983, 113, 173), EMS showed borderline cytotoxicity at about 10 mg/mL concentration in DMSO; when compared with DGEBA at 10 mg/mL DMSO, EMS was about one-tenth (1/10th) as cytotoxic as DGEBA. Its advantages over the state of the art are: (a) ability to cure very rapidly at relatively low temperatures with olefins amines, polyamines, thiols, polythiols, polyols, carboxylic acids, anhydrides, isocyanates, amides, polyamides, poly amino amides, and the like; (b) negligible cytotoxicity and mutagenicity; and (c) it contains from one to eight methallyl and from one to six epoxy substitutions, as desired for final applications.

One primary object of this invention is to provide a sucrose-based epoxy monomer having the ability to cure faster than other sucrose-based epoxy monomers and other epoxies of more traditional composition.

Another primary object of the invention is to provide a sucrose-based monomer having terminal double bonds that will have at least one moderately active olefin towards epoxidation.

Another primary object of the invention is to provide a sucrose-based epoxy monomer having terminal double bonds and terminal epoxy groups that cure with olefins; epoxies; amines, polyamines, polyaminoamides, and polyamides; isocyanates and urethanes; thiols, polythiols, sulfides and polysulfides; carboxylic acids, polycarboxylic acids, amino acids, polyamino acids, anhydrides and polyanhydrides; and alcohols and polyols upon thermal, electrophilic and/or nucleophilic activation.

Another primary object of the invention is to provide a sucrose-based monomer having epoxy groups that, at room temperature, are more reactive than other sucrose-based epoxy monomers and other epoxies of more traditional composition.

Another primary object of the invention is to provide a sucrose-based monomer having epoxy groups that, at sub-ambient temperatures, are readily reactive with nucleophiles and electrophiles.

Another primary object of the invention is to provide a sucrose-based monomer having, on average, at least 5 epoxy groups per sucrose.

Another primary object of the invention is to provide a sucrose-based monomer having, when cured with amines, polyamines and polyaminoamides, an average peak curing temperature of about 75° C.

Another primary object of the invention is to provide a sucrose-based epoxy monomer that when cured with anhydrides in the presence of catalytic amounts of tertiary amines exhibits an average peak curing temperature of about 75° C.

Another primary object of the invention is to provide a sucrose-based epoxy monomer that, when cured with thiols, polythiols, amino acids, polyamino acids and thiol-containing amino acids has an average peak curing temperature of ˜50° C.

Another primary object of the invention is to provide a sucrose-based epoxy monomer that is either non-cytotoxic or less cytotoxic than traditional commercial epoxies.

Another primary object of the invention is to provide a sucrose-based epoxy monomer that is either non-mutagenic or less mutagenic than traditional epoxies.

Another primary object of the invention is to provide a sucrose-based monomer having highly reactive epoxy groups capable or reacting at room or ambient temperatures.

Another primary object of the invention is to provide a sucrose-based epoxy monomer that readily reacts with nucleophiles at sub-ambient temperatures.

Another primary object of the invention is to provide a sucrose-based epoxy monomer that generates tough thermosets with moderate glass transition temperatures.

Another primary object of the invention is to provide a sucrose-based ether monomer having high yields when prepared in aqueous or organic media.

Another primary object of the invention is to provide saccharide-based ethers and epoxy ethers that are liquids at or below room temperature.

Another primary object of the invention is to provide a saccharide-based epoxy having a tensile strength that is comparable or higher than that of DGEBA.

Another primary object of the invention is to provide a saccharide-based monomer that results in tough adhesives, coatings, and composites.

It is another object of the invention to provide monomers and prepolymers using a saccharide monomer, in particular a saccharide-based monomer having one to eight methallyl groups and at least one hydroxyl group, preferably a long chain (C₄-C₂₀) methallyl-containing ether group on the hydroxyl group. More preferably, the long chain (C₄-C₂₀) methallyl-containing ether group will have more than one double bond in the carbon chain.

A further aspect of the present invention is a method for producing a saccharide-based epoxy monomer having one to eight epoxy groups per molecule of sucrose (with an average of five to six epoxy groups per sucrose molecule), which comprises reacting a mixture comprising: a monomer comprising a methallyl ether-containing group which is bonded to at least one primary or secondary hydroxyl group on a saccharide; and an enzyme, acid, organometal reagent, or metal reagent or catalyst in the presence of an oxidizing agent, in relative amounts sufficient to produce a saccharide-based epoxy monomer having one to eight epoxy groups per molecule of sucrose (with an average of five to six epoxy groups per sucrose).

Preferably, the methallyl-containing group of the sucrose monomer is a C₄-C₂₀ methallyl-containing ether. More preferred is a C₄-C₂₀ methallyl-containing ether that may contain more than one double bond to provide more sites for epoxidation and subsequent crosslinking.

Further, the present invention provides a crosslinked resin produced from reacting a mixture comprising: methallylsucrose-based epoxy monomers having one to eight epoxy groups per molecule of sucrose; and a curing agent, in relative amounts sufficient to produce a saccharide-based crosslinked resin. In such methods, the curing agent may be a nucleophilic curing agent or an electrophilic curing agent.

In another aspect of the present invention, saccharide-based epoxy monomers are used to produce epoxy resins. In resins, the saccharide-based epoxy monomers may comprise more than 50% of the weight of the polymer. Furthermore, saccharide-based epoxy resins may be copolymerized with known epoxy materials to generate resins with less than 50% by weight of the saccharide-based materials.

In another aspect of the present invention, the crosslinked resin produced by such methods are provided.

Other objects of the invention will become apparent from a full review of this application.

### (G) BRIEF DESCRIPTION OF THE SEVERAL VIEWS OF THE DRAWINGS

FIG. 1 is a schematic depiction of monomers of EAS.

FIG. 2 is a schematic depiction of monomers of EMS.

FIG. 3 is a schematic depiction of monomers of ECS.

FIG. 4 is a schematic depiction of monomers of DGEBA.

FIG. 5 is an ¹H-NMR spectrum of MS.

FIG. 6 is an ¹H-NMR spectrum of EMS.

FIG. 7 is a ¹³C-NMR spectrum of MS.

FIG. 8 is a ¹³C-NMR spectrum of EMS.

FIGS. 9(a) through (f) depict a group of graphs comparing the cytotoxicity and mutagenicity potentials of EAS, EMS, ECS and DGEBA using the modified Maron-Ames test.

FIG. 10 is a graph depicting the Dynamic Mechanical Analysis (DMA) scans of EAS, EMS, ECS and DGEBA; the vertical axis indicates elastic modulus (E′, Pa), while the horizontal axis indicates temperature (° C.).

FIG. 11 depicts DMA scans of UNI-REZ 2142 (40 and 70 phr) cured epoxies.

FIG. 12 depicts DMA scans of UNI-REZ 2355 (40 and 70 phr) cured epoxies.

FIG. 13 depicts the structures of representative saccharide-based polyols.

FIG. 14 depicts the conversion of representative saccharide-based polyols using the anhydrous method disclosed herein.

FIG. 15 depicts the conversion of representative saccharide-based polyols using the aqueous method disclosed herein.

FIG. 16 is a schematic depiction of conversion of a cellulose using the method disclosed in Sachinvala et. al, J. of Poly. Sci: Part A: Poly. Chem. Ed. 2000, 38, 1889-1902, incorporated herein by reference.

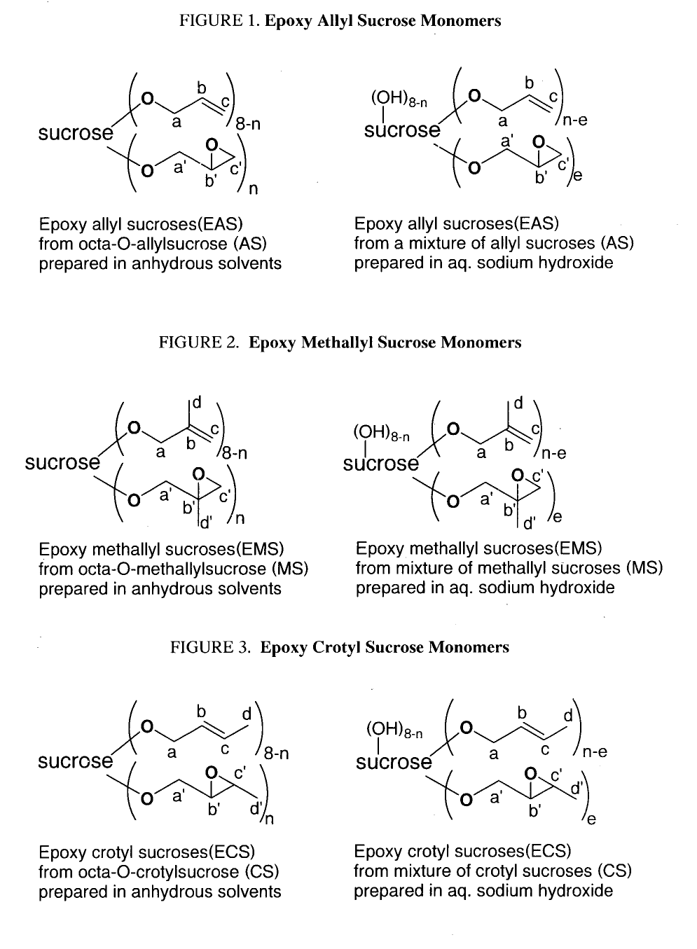

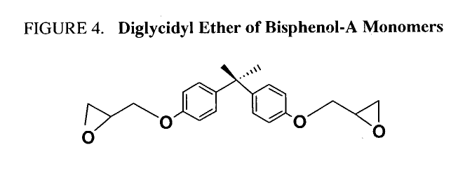

Table 1 shows temperature dependent density measurements for the sucrose-based monomers and their Arrhenius equations.

Table 2 shows the dynamic viscosities (flow under the influence of gravity) of the monomers.

Table 3 shows Tg data from neat sucrose-based monomers by DSC and DMA.

Table 4 charts the ratios for test mixes of sucrose-based epoxidized monomers (or DGEBA) with curing agents.

Table 5 summarizes the DSC data for the test mixes charted in Table 4.

Table 6 summarizes the DMA results for the test mixes charted in Table 4.

Table 7 summarizes the results of adhesion studies on EAS, ECS and DGEBA.

Table 8 summarizes the results of adhesion studies on EMS.

These drawings and tables illustrate certain details of certain embodiments. However, the invention disclosed herein is not limited to only the embodiments so illustrated. The invention disclosed herein may have equally effective or legally equivalent embodiments.

### (H) DETAILED DESCRIPTION OF THE INVENTION

The claims of this invention are to be read to include any legally equivalent composition of matter or method. Before the present invention is described in detail, it is to be understood that the invention is not limited to the particular configurations, process steps and materials disclosed herein.

For the sake of simplicity and to give the claims of this patent application the broadest interpretation and construction possible, the following definitions will apply to this application:

1. The term “saccharide-based” or derivative thereof means any mono-saccharide, di-saccharide, tri-saccharide, oligo-saccharide, any polysaccharide without a reducing (aldehyde, ketone, or acetal) end, any saccharide from which the reducing end has been removed by reduction to yield a polyol, any saccharide from which a reducing end has been converted to an acetal, or any saccharide whose reducing end bears no significance to the final product.

2. The word “monomer” or derivative thereof includes prepolymers having at least one reactive group functioning like a monomeric reactive group.

3. The acronym “MS” means methallyl sucrose, an unepoxidized monomer containing methallyl (or 2-methyl-2-propenyl) pendants.

4. The acronym “EMS” means epoxymethallyl sucrose, an epoxidized monomer containing methallyl (or 2-methyl-2-propenyl) pendants as well as 2-epoxy-2-methyl-propyl pendants (commonly known epoxy methallyl) or (if the context suggests) a polymer containing EMS.

5. The acronym “AS” means allyl sucrose, an unepoxidized monomer containing 2-propenyl pendants.

6. The acronym “EAS” means epoxyallyl sucrose, an epoxidized monomer containing 2-propenyl as well as 2-epoxypropyl pendants, commonly known as epoxy allyl, or (if the context suggests) a polymer containing EAS.

7. The acronym “CS” means crotyl sucrose, an unepoxidized monomer containing 2-butenyl pendants.

8. The acronym “ECS” means epoxycrotyl sucrose, an epoxidized monomer containing 2-epoxy butyl pendants (commonly known as epoxy crotyl) or (if the context suggests) a polymer containing ECS.

9. The phrase “nucleophillic curing agent” means a substance that accelerates the reaction of nucleophiles with molecules having epoxy groups; examples include (without limitation) tertiary amines (such as triethylamine and tributylamine) and tertiary phosphenes (such as tributylphosphenes and triphenolphosphenes).

10. The phrase “electrophillic hardener/catalyst” means a substance that accelerates the reaction of electrophiles with molecules having epoxy groups, including a Lewis acid that activates the polymerization of epoxy groups to result in a polyether.

11. The phrase “long chain hydrocarbon,” or derivative thereof means a hydrocarbon ether pendant attached to a saccharide hydroxyl group via etherification, esterification or acetal formation, and the hydrocarbon chain contains one or more double bonds for epoxidation; such hydrocarbon pendant may create void volumes in the polymer that improve impact performance.

In most general form, the invention disclosed herein comprises a saccharide monomer containing a methallyl ether on at least one hydroxyl group; see MS, below, obtained from anhydrous and aqueous preparations, respectively.

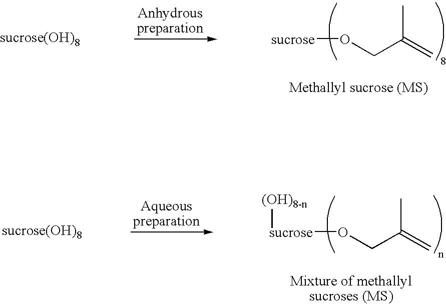

The present invention also includes an epoxidized monomer having an average of least 5 epoxy groups per saccharide molecule. One depiction of one particular version of the epoxy monomer is comprised of:

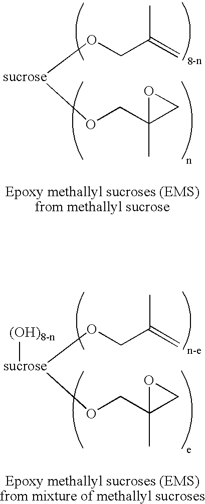

The invention disclosed herein also relates to the polymerization of such epoxy monomers to form a thermoset (polymer) when the monomer is treated with (A) a curing agent comprising an amine, polyamine, amidoamine, polyaminoamide, thiol, polythiols, polysulfides, carboxylic acid, anhydride, polycarboxylic acids, polyanhydrides, amino acid, polyamino acids; (B) with or without nucleophilic activating agent or agents such as trialkylamines, triarylamines, triallylamines, epoxy allyl amines, trialkylphosphines, triarylphosphines, triallylic phosphines, epoxy allyl phosphines, aminoalcohols, aminophenols, and the like; or (C) with or without electrophilic activating agents such as boron halides, aluminum halides, alkyl-aluminum reagents, alkyl-aluminurn halides, other Lewis acids, and the like. Such thermoset formation may occur at or below room temperature, as well as upon application of heat. Moreover, the present invention includes a method of making said unepoxidized monomers, epoxidized monomers, polymerized thermosets, and resins including the same.

In one general version of the invented method, EMS was prepared by a two-step process, involving: (a) methallylation of sucrose to MS, using aqueous sodium hydroxide and methallyl chloride; and (b) epoxidation of the mixture of methallyl sucroses (MS) with peracetic acid to produce EMS.

Alternatively, octa-O-methallylsucrose was prepared by treatment of a solution of sucrose in DMSO, DMAc, DMF, or pyridine, with sodium hydride, followed by addition of methallyl chloride. MS obtained by this method was then epoxidized to form EMS using peracetic acid.

More particularly, in the first step yielding MS monomers, for example, sucrose (200 g, 0.584 mol, 4.674 mol hydroxyl groups) and aqueous NaOH (280.5 g in 138 mL water, 7.011 mol, 1.5 eq./hydroxyl group) were added to a Parr pressure vessel. The vessel was sealed, heated with stirring to between 80° C. and 100° C. over 30 minutes, and maintained at that temperature for about an hour to dissolve the reagents. The contents were then cooled to ˜50° C., the vessel opened, and charged with cold methallyl chloride (693 mL, 7.011 mol, 1.5 equiv./hydroxyl group) in one portion. The reactor was then sealed and pressurized with nitrogen gas (˜100 PSI). The internal temperature was equilibrated to about 80° C. over about two hours, and the contents were stirred overnight. Subsequently, the vessel was cooled to room temperature, placed in an ice bath, depressurized, opened, and diluted with ice water (500 mL) to dissolve the salts. The contents were transferred to a separatory funnel with ice water. Additional ice water was added to the separatory funnel, and the mixture was extracted with cold ethyl acetate (2×250 mL). The combined organic layers were then washed serially with water (1×300 mL) and brine (1×500 mL), dried over sodium sulfate, filtered, and concentrated in vacuo (0.1 mm Hg, 40-50° C., overnight). MS (375 g, 0.529 mol) was obtained in 90.4% yield. The average degree of methallyl substitution was 6.8 (DS=6.8, by NMR).

In step two yielding EMS, a three-neck Morton flask, fitted with a high torque overhead mechanical stirrer, pressure-equalized addition funnel, and a condenser connected to a nitrogen gas line, was placed in a refrigeration bath. The flask was charged with, for example, MS (average molecular weight 709.5, 500 g, 0.705 mol, 4.79 mol double bonds) dissolved in ethyl acetate (1.0 L), and sodium acetate (47.17 g, 0.575 mol, 10% of the number of moles of peracetic acid) was then added to the solution. The contents were cooled to about 5° C., and peracetic acid (32% in acetic acid, d=1.13 g/mL, 1.209 L, 5.751 mol) was added dropwise into the mixture over about two hours. The temperature was then raised to about 10° C., and the contents stirred overnight. Subsequently, the mixture was diluted with ethyl acetate (2 L), transferred to a separatory funnel, and washed serially with cold water (2×500 mL), cold aqueous saturated sodium carbonate (1×500 mL), and brine (2×500 mL). The organic layer was then separated, dried over anhydrous sodium carbonate, filtered, and concentrated in vacuo (0.1 mm Hg, 50° C., about 1 hour) to yield EMS as an oil that is clear and light yellow in appearance, in 93% yield (524.3 g, 0.656 mol.). No further purification was required.

The present invention also includes a method for preparing an epoxymethallyl saccharide monomer comprising treating a dimethylacetamide or dimethylsulfoxide solution of sucrose (for example) with a suspension of dimethylsulfoxide and sodium hydride at temperature of about 10° C. to obtain a sodium sucrate mixture. The sucrate mixture is then stirred for about 80 to 90 minutes, while allowing the temperature to attain between about 30° C. to 40° C.; the mixture is then stirred for about 60 minutes at that temperature. The method further comprises cooling the sucrose mixture to between about 0° C. and 10° C., and treating the sucrose mixture with methallyl chloride (1.2 equivalent per mole hydroxyl groups). Following addition at about 10° C., the temperature of the reaction mixture was monitored internally and the mixture allowed to attain about 40° C. to 50° C., and stirred overnight. The resulting yellow mixture was cooled to about 10° C. and quenched with 5% aqueous sodium hydroxide, then diluted with water and extracted with ethyl acetate. The organic extracts were combined, washed serially with water and brine, dried over anhydrous sodium sulfate, filtered through charcoal and then concentrated in vacuo. Flash column chromatography of the residue, on a silica gel column using hexanes and 10% ethyl acetate in hexanes provided the desired octa-O-methallylsucrose in 87 to 94% yield.

Preferably, a dimethylacetamide (DMAc) or dimethylsulfoxide (DMSO) solution of sucrose (5 g, 14.62 mmol in 30 mL solvent) was treated with a suspension of DMAc or DMSO (300 mL) and sodium hydride (60% in oil, 8.4 g, 210 mmol, washed four times with 15 mL of dry hexane). To the sodium sucrate mixture at 10° C. was added methallyl chloride (14.6 mL added over 30 minutes), the temperature equilibrated to about 50° C., and the contents stirred for about 90 minutes. Later the contents were cooled to about 10° C., quenched with 5% aqueous sodium hydroxide (30 mL), diluted with water (500 mL) and extracted with ethyl acetate (4×100 mL). The organic extracts were combined, washed serially with water and brine (3×150 mL each), dried over anhydrous sodium sulfate, filtered through charcoal and then concentrated in vacuo. Flash column chromatography of the residue on a silica gel (230-440 mesh) column (diameter×length=7 cm×15 cm) using hexanes and 10% ethyl acetate in hexanes (3L) provided the desired products in 87-94% yields.

All of the above are relative amounts only, and may be proportionately altered in practicing the invention. The invention should not be limited by the stated exemplary amounts.

As discussed above, the subject invention in one of its preferred embodiments relates to methods of using saccharide-based monomers having a methallyl group on at least one of the hydroxyl groups, preferably a long chain (C₄-C₂₀) methallyl-containing ether on the hydroxyl group, and more preferably, the long chain (C₄-C₂₀) methallyl-containing ether having more than one double bond in the pendant carbon chain.

Generally, monomers requiring anhydrous preparation were prepared in dry glassware under an inert atmosphere, using conditions described in Sachinvala, N. D. et al., Carbohydrate Research, 1991, Vol. 218, pp. 237-245. Etherifications requiring aqueous conditions were effected in aqueous media in a pressure reactor pressurized using nitrogen or argon gas. Proton nuclear magnetic resonance (NMR) spectra were recorded at 500.11 MHz, and carbon-13 NMR spectra were recorded at 125.76 MHz. using a General Electric GN Omega 500 spectrometer. Fast atom bombardment (FAB) mass spectra were obtained on a VG instrument (Model 70 S.E.) using xenon as a bombarding gas. Molecular ions were verified as [M+1]⁺, [M+K]⁺ or [M+Na]⁺by addition of potassium or sodium iodide to the sample matrix. All organic reagents and solvents (reagent grade, Aldrich Chemical Company) used in monomer syntheses were purified and dried before use according to procedures outlined by Perrin et.al. (Purification of Laboratory Chemicals, 2^{nd} edition, Pergamon Press, Oxford, 1990). Flash column chromatography was performed according to Still et al. (J. Org. Chem., 1978, Vol. 43, pp. 2923-2925). Elemental analyses were performed by Desert Analytics (Tucson, Ariz.).

The above-described sucrose derivatives can be partly converted to methallyl ethers and then epoxidized to epoxymethallyl ethers upon treatment with an acidic or metallic catalyst in the presence of an oxidizing agent. In a preferred embodiment, the sucrose monomer is 1′,2,3,3′,4,4′,6,6′-octa-O-methallylsucrose. For the conversion process, an acidic or metallic catalyst together with an oxidizing agent is added to a monomer comprising a methallyl-containing group which is bonded to at least one primary or secondary hydroxyl group on a sucrose, in relative amounts sufficient to produce a sucrose-based epoxy monomer having one to eight epoxy groups per molecule of sucrose. Preferred acidic and metallic catalysts include peracid, molybdenum, tungsten, and vanadium catalysts; more preferred catalysts include peracid and traditional molybdenum hexacarbonyl and phosphotungstic acid oligomers. Preferred oxidizing agents include hydrogen peroxide, t-butyl hydroperoxide and derivatives thereof. More preferred oxidizing agents include hydrogen peroxide, t-butyl hydroperoxide, and derivatives thereof.

More specifically, epoxidation may be effected by use of enzymes (lipases) in the presence of a carboxylic acid and hydrogen peroxide. The enzymes oxidize the carboxylic acid to the peroxy acid, which in turn epoxidized the olefin. A discussion of such enzyme systems may generally be found, for example, in F. Bjorkling et.al., J. Chem. Soc., Chem. Commun., 1990, 1301; E. Santaniello et.al., Chem. Rev., 1992, 92,1071; K. Faber et.al. Synthesis, 1992, 895; F. Bjorkling et al, Tetrahedron, 1992, 48, 4585; T. Mashino et. al. Tetrahedron Lett., 1990 31, 3163; H. Fuet al, J. Am. Chem. Soc., 1991, 113 5878; and 0. Takahashi et. al., Tetrahedron Lett., 1989, 30, 1583. These references are hereby incorporated by reference.

Peracids have also been traditionally used to transform olefins to epoxides. Commonly used peracids include peracetic acid, peroxyimidic acids, meta-chloroperbenzoic acid and magnesium peroxyphthalate. References for various peroxy acids that have been used to effect olefin epoxidation may be found, for example, in Comprehensive Organic Transformation (VCH, New York, 1989, pp. 456-459). This reference as well as those cited therein is incorporated by reference.

Tungstic acid reagents may also be employed for epoxidation. Treatment of sodium tungstate with phosphoric acid produces phosphotungstic acid oligomers. These compounds in the presence of excess hydrogen peroxide form peroxy tungstides that readily epoxidize olefins. The reagent is effective even with terminal olefins. Typically, these reactions are performed under phase transfer conditions as described in Fort et. al., Tetrahedron, 1992, 48, 5099-5110; Venturello et. al., J. Org. Chem., 1983, 48, 3831-3833; Quenard et. al., Tetrahedron Lett., 1987, 2237-2238; and Prandi et. al., Tetrahedron Lett., 1986, 2617-2620. These references are also incorporated by reference.

Other catalysts may also be used to effect epoxidation. For example, in the presence of hydrogen peroxide or alkyl hydroperoxides, tungsten, vanadium and molybdenum compounds catalytically convert olefins to epoxides in non-polar organic solvent or aqueous organic biphases. Such reactions are set forth in, for example, Sharpless et.al., J. Am. Chem. Soc., 1973, 95,6136; Itoh et.al., Chem. Comm., 1976, 421-423; Rajan et.al., Tetrahedron, 1984, 40, 983-990; Antoniolette et.al., J. Org. Chem., 1983, 48, 3831-3833; and Mihelich et.al., J. Am. Chem. Soc., 1987, 103, 7690-7692).

As shown in FIG. 2, octa-O-methallylsucrose is epoxidized to produce the mixed MS derivative, wherein the average number of epoxy groups per molecule of sucrose is about 5 or 6. In general, the number of epoxy groups can vary from 1 to 8 per molecule of sucrose. As previously stated, this epoxidation reaction may be achieved, for example, by a variety of acidic or metallic catalysts such as peracids (see Hudliky, M. Oxidation in Organic Chemistry, A.C.S. Monograph 186, American Chemical Society, Washington D.C. 2505-2511), and oligmers of phospho tungstic acid (see, Venturello, C.; Aloisio, R., J. Org. Chem., 1988, 53, 1553-1557), in the presence of such oxidizing agents as t-butyl hydroperoxide, or hydrogen peroxide.

The two-step conversion process of sucrose to epoxy monomers produces at least two products. In the first step, octa-O-methallylsucrose is produced. In the second step, octa-O-methallylsucrose is converted to one of several isomeric sucrose-based epoxy compounds. The resulting epoxy monomers from octa-O-substituted methallyl will have a range of 1-8 epoxy groups per sucrose monomer, and an average number of epoxy groups per sucrose of about 5.5. These monomers may be cured, i.e., crosslinked, to then produce sucrose-based polymeric epoxy resins.

A second group of products from the two-step conversion of sucrose to epoxy resins are epoxides produced from partially-O-methallylated sucroses. These epoxy products will have different polarities than the fully substituted monomers and should, therefore, find different applications. These epoxies will also be less expensive to produce.

For use in structural and coating applications, for example, the saccharide-based epoxy compounds may be reacted with curing agents generally known in the art to produce a crosslinked resin. Curing agents are co-reactants that attack and open the epoxide ring in the crosslinking (curing) process. Curing agents useful in the present invention include both nucleophilic and electrophilic curing agents. Nucleophilic curing agents include ureas, urethanes, amines, thiols, phenols, amides, ketimines, sulfides, mercaptans, acids and imidazoles. (see, Tanaka et.al., in Epoxy Resins: Chemistry and Technology, 2^{nd} Edition, May, C. A. (editor), Marcel Dekker, New York. 1988, pp 285-463). Examples of amine curing agents include diamines, polyamines, dicyanodiamide, and aminoplasts (see, Tanaka et.al., in Epoxy Resins: Chemistry and Technology, 2nd Edition, May, C. A. (editor), Marcel Dekker, New York, 1988, pp 285-463; Mika and Technology, 2^{nd} Edition, May. C. A. (editor), Marcel Dekker, New York, 1988, pp 285-463; Mika et.al., R. S. idem, pp 465-550). Amines, diamines and polyamines are preferred curing agents since each nitrogen to hydrogen bond is potentially capable of reacting with an epoxy group to increase the density of crosslinking. Particularly preferred amines are selected from triethylenetetramine, dicyandiamide and aminoplasts.

Thiols, polysulfides and poly-mercaptans are also preferred in certain applications for producing fast curing epoxy resins and adhesives. These curing agents attack the epoxide ring at the least hindered site, to open the ring and crosslink (cure) the system. The sulfides and thiols bind metal surfaces and impart excellent adhesive properties to the resins (see, Tanaka et.al., in Epoxy Resins: Chemistry and Technology, 2^{nd} Edition, May, C. A. (editor), Marcel Dekker, New York, 1988, pp 285-463).

Phenolic and phenoplast resins open the epoxy group in the presence of a strong acid (catalyst) and cure via the hydroxy group at high temperatures. Such curing resins are useful for high temperature applications (see, Tanaka et. al., in Epoxy Resins: Chemistry and Technology, 2^{nd} Edition, May, C. A. (editor), Marcel Dekker, New York, 1988, pp 285-463).

Acidic curing agents include carboxylic acids and their anhydrides. These curing agents will react with the epoxy group with heating (see, Tanaka et. al., in Epoxy Resins: Chemistry and Technology, 2^{nd} Edition, May, C. A. (editor), Marcel Dekker, New York, 1988, pp 285-463).

Electrophilic curing agents may also be used. Such curing agents include, e.g., latent acid catalysts, aryl iodonium salts, aryl-sulfonium salts and aryl selenium compounds. Latent acid catalysts thermally or photochemically generate acid complexes, promote ring opening and polymerization of the epoxide by acid catalysis. Aryl iodonium and aryl-sulfonium salts contain stable anions that photochemically release protic acids. The protic acids then catalyze epoxy ring opening polymerization, to produce thin coats of epoxy resins on metal surfaces (see, Tanaka et.al., in Epoxy Resins: Chemistry and Technology, 2 Edition, May, C. A. (editor), Marcel Dekker, New York, 1988, pp 285-463; Mika, T. F.; Bauer, R. S. idem, pp 465-550).

In general, the preferred curing agents include di-polyamines and tri-polyamines.

One general embodiment of the invention disclosed herein includes (comprises) a composition of matter, comprising an unepoxidized monomer. Said unepoxidized monomer comprises (includes) a non-sucrose saccharide-based molecule comprising at least one methallyl-containing group bonded to at least one hydroxyl group thereof. More particularly, said bond may be between said methallyl-containing group and at least one primary or secondary hydroxyl group. Moreover, said methallyl-containing group comprises a long chain methallyl-containing ether group on said hydroxyl group.

In a more particular embodiment, said long chain methallyl-containing ether comprises more than one double bond in the carbon chain. Another embodiment includes said non-sucrose saccharide-based molecule being fully substituted with methallyl-containing groups, each bonded to at least one different hydroxyl group thereof.

Specific embodiments of the unepoxidized monomer may include 1,2,3-tri-O-methallyl glycerol; 1,2,3,4,5-penta-O-methallyl xylitol; 1,2,3,4,5,6-hexa-O-methallyl sorbitol; 1,2,3,4,5,6-hexa-O-methallyl mannitol; or a cellulose derivative wherein each of its hydroxyl groups are substituted with at least one methallyl-containing group.

The invention disclosed herein may also include a composition of matter, comprising a saccharide-based epoxy monomer having at least one geminally disubstituted terminal epoxy group per saccharide and at lease one geminally disubstituted terminal double bond. More particularly, said epoxy monomer may include saccharide-based epoxy monomer wherein said epoxy comprises epoxymethallyl glycerol, more particularly, 1,2,3 tri-O-epoxy methallyl glycerol. In another embodiment, said epoxy comprises epoxymethallyl xylitol, more particularly, 1,2,3,4,5-penta-O-epoxymethallyl xylitol. Said epoxy monomer may also include epoxymethallyl sorbitol, more particularly, 1,2,3,4,5,6-hexa-O-epoxymethallyl sorbitol. In another embodiment, said epoxy includes epoxymethallyl mannitol; more particularly, 1,2,3,4,5,6-hexa-O-epoxymethallyl mannitol. In another embodiment, said epoxy includes epoxymethallyl sucrose; more particularly, 1′,2,3,3′,4,4′,6,6′-octa-O-epoxymethallyl sucrose. In another embodiment, said epoxy includes epoxymethallyl cellulose.

The invention disclosed herein may also include a composition of matter comprising a polymerized epoxy mixture comprising a plurality of saccharide-based epoxy monomers as described above, and a curing agent. Said curing agent may be a nucleophilic hardener such as (for example) one or more selected from the group consisting of ureas, urethanes, amines, thiols, phenols, amides, ketimines, sulfides, mercaptans, amino acids and imidazoles, and mixtures thereof.

For one such epoxy mixture, said hardener is selected from the group consisting of amines, diamines and polyamines, and mixtures thereof. For another such epoxy mixture, such hardener is selected from the group consisting of diethylenetriamine, triethylenetetramine, tetraethylenepentamine, dicyandiamide and aminoplasts, and mixtures thereof. For another such epoxy, said hardener is selected from the group consisting of thiols, polysulfides and polymercaptans, and mixtures thereof.

In one such epoxy mixture, said saccharide-based epoxy monomers are epoxymethallyl sucroses, and said curing agent is selected from the group consisting of polyamides, polyamidoamines, diethylenetriamines and mixtures thereof. In another such epoxy mixture, said saccharide-based epoxy monomers are epoxymethallyl xylitols, and said curing agent is selected from the group consisting of polyamides, polyamidoamines, diethylenetriamines and mixtures thereof. In another such epoxy mixture, said saccharide-based epoxy monomers are epoxymethallyl mannitols, and said curing agent is selected from the group consisting of polyamides, polyamidoamines, diethylenetriamines and mixtures thereof. In yet another such epoxy mixture, said saccharide-based epoxy monomers are epoxymethallyl celluloses, and said curing agent is selected from the group consisting of polyamides, polyamidoamines, diethylenetriamines and mixtures thereof.

Alternatively, said curing agent may be an electrophilic hardener/catalyst. For one type of epoxy, said hardener/catalyst is selected from the group consisting of latent acid catalysts, borontriflourideethylamine complex, aryl iodonium salts, aryl sulfonium salts and aryl selenium compounds, and mixtures thereof.

The invention disclosed herein may also include an adhesive comprising an epoxy mixture wherein:

a. said saccharide-based epoxy monomers are selected from the group consisting of epoxyallyl sucrose, epoxycrotyl sucrose, epoxymethallyl sucrose, epoxymethallyl sorbitols, epoxymethallyl xylitols and epoxymethallyl celluloses, and mixtures thereof; and

b. said curing agent is selected from the group consisting of aliphatic and aromatic polyamines, polyamides, polyamidoamines, polythiols, polymercaptans and amino acids, and mixtures thereof.

In one such adhesive, said saccharide-based epoxy monomers are selected from the group consisting of epoxymethallyl sucroses, epoxymethallyl xylitols, epoxymethallyl mannitols and epoxymethallyl celluloses, and mixtures thereof. Said curing agent is selected from the group consisting of polyamides, polyamidoamines, diethylenetriamines and mixtures thereof.

The epoxies of the present invention may be useful in making adhesives that cure relatively quickly at relatively low temperatures, including so-called ambient or room temperatures in the range of about 20° C. to about 25° C. In one such adhesive, said saccharide-based epoxy monomers are epoxymethallyl saccharides, and said curing agent is selected from the group consisting of diethylenetriamines, triethylenetetramines, tetraethylenepentamines and thiols, and mixtures thereof.

The epoxies of the present invention may also be useful in making an adhesive that is only temporarily adhesive, or that releases its adhesion over the passage of time (especially as a consequence of exposure to heat such as, for example, hot water). In one such adhesive, said saccharide-based epoxy monomers are a mixture of epoxyallyl saccharides and said curing agent is polyamides or polyamidoamines.

The invention disclosed herein may also include a coating comprising an epoxy mixture of, and further comprising a viscosity modifier. Preferably, said viscosity modifier is selected from the group consisting of methylethylketone, 2-pentanone, cyclohexanone, xylene, cresol and ethyleneglycoldimethylether, and mixtures thereof. In one such coating, said saccharide-based epoxy monomers are selected from the group consisting of epoxyaltyl sucrose, epoxycrotyl sucrose, epoxymethallyl sucrose, epoxymethallyl sorbitols, epoxymethallyl xylitols and epoxymethallyl celluloses, and mixtures thereof. Said curing agent is selected from the group consisting of aliphatic and aromatic polyamines, polyamides, polyamidoamines, polythiols, polymercaptans and amino acids, and mixtures thereof; and said viscosity modifier is selected from the group consisting of solvents that dissolve and lower the viscosity of the amine curing agent, and mixtures thereof. In one type of such coating, said saccharide-based epoxy monomers are epoxymethallyl sucrose, said viscosity modifier is methylethylketone, and said curing agent is selected from the group consisting of metaxylenediamine, diethylenetriamine and polyamidoamine, and mixtures thereof.

More particularly, in one such coating, said saccharide-based epoxy monomers are epoxymethallyl sucrose, said curing agent is metaxylenediamine, and said viscosity modifier is methylethylketone; this coating cures to a hard thermoset.

The invention disclosed herein may also include a composite material comprising a saccharide-based epoxy and further comprising a filler. Said filler may be vegetable matter such as (for example) material selected from the group consisting of bagasse, kenaf, nonwoven cotton, wheat straw, rice hull, bamboo, defoliated plant matter and sawdust, and mixtures thereof.

One particular embodiment includes a composite that is load bearing and/or hard. One such composite includes concrete filler, wherein: said saccharide-based epoxy monomers are selected from the group consisting of epoxycrotyl and epoxymethallyl saccharide-based monomers, and mixtures thereof; said curing agent is selected from the group consisting of amines, polyamines, amides, polyamides, thiols, polythiols, polymercaptans, anhydrides, amino acids and polyanhydrides, and mixtures thereof; and other filler is selected from the group consisting of bagasse, kenaf, wheat straw, rice hull, bamboo, defoliated plant matter and sawdust, and mixtures thereof.

In one particular embodiment of the composite including concrete filler, said saccharide-based epoxy monomers are 1′,2,3,3′,4,4′,6,6′-octa-O-epoxymethallyl sucrose, said curing agent is selected from the group consisting of diethylenetriamine and methylene-phylene diamene, and said fillers are selected from the group consisting of bagasse, kenaf and defoliated cotton plant, and mixtures thereof. In another particular embodiment, said saccharide-based epoxy monomers are selected from the group consisting of 1′,2,3,3′,4,4′,6,6′-octa-O-epoxymethallyl sucrose and 1′,2,3,3′,4,4′,6,6′-octa-O-crotyl sucrose (and mixtures thereof), said curing agent is norbornadiene anhydride in the presence of a catalytic amount of a catalyst selected from the group consisting of trioctylamine and metaxylenediamine (and mixtures thereof), and said fillers are selected from the group consisting of bagasse, kenaf and defoliated cotton plant (and mixtures thereof).

The invention disclosed herein may also include composites that are more flexible, moldable, or that have insulative qualities. In one such embodiment, said saccharide-based epoxy monomers are epoxyallyl saccharides, said curing agent is selected from the group consisting of polyamidoamines and polythiols (and mixtures thereof), and said filler is long fiber matter. In one particular embodiment, said filler is selected from the group consisting of nonwoven cotton, nonwoven kenaf, nonwoven jute and fiberglass (and mixtures thereof). Another particular embodiment further includes polyester and polypropylene; preferably, the ration of said filler(s) to the polyester and polypropylene is about 1:1:1.

Besides the aforementioned compositions of matter, the present invention includes methods of making them. One general method of making an unepoxidized monomer disclosed above includes the steps of reacting a saccharide-based monomer in aqueous sodium hydroxide with methallyl chloride. More particularly, said method may include the steps of:

a. combining said saccharide-based monomers and aqueous NaOH in a vessel, and heating for about one hour and thirty minutes;

b. cooling same, and adding cold methallyl chloride;

c. equilibrating the internal temperature of same, and stirring;

d. placing said vessel in an ice bath, depressurizing same, and diluting said contents with ice water;

e. extracting organic contents with cold ethyl acetate;

f. washing said extraction, drying same, filtering it, and concentrated same in vacuo.

Another embodiment of the method includes the steps of:

a. combining about 0.584 moles of sucrose in about 7.011 moles of aqueous NaOH in sealed pressure vessel, heating to the range of between about 80° C. and 100° C. for about thirty minutes, and maintaining said temperature for about one hour;

b. cooling the contents of said vessel to about 50° C., and adding about 7.011 moles cold methallyl chloride, then pressurizing said vessel with nitrogen gas;

c. equilibrating the internal temperature of said vessel to about 80° C. over two hours, and stirring the contents for about overnight;

d. cooling said vessel to about room temperature, placing said vessel in an ice bath, depressurizing said vessel and diluting said contents with ice water;

e. transferring said contents to a separatory funnel with ice water, and extracting an organic layer of same with cold ethyl acetate;

f. washing serially said extraction with water and brine, drying same over sodium sulfate, filtering same, and concentrating same in vacuo overnight.

Another method of making unepoxidized monomers includes using about 0.779 moles of sorbitol in place of sucrose. Another method includes using about 0.935 moles of xylitol in place of sucrose.

An alternative method of making an unepoxidized monomer includes the steps of reacting a saccharide-based monomer in sodium hydride in dimethylsulfoxide with methallyl chloride. One general method of making an unepoxidized monomer disclosed above includes the steps of:

a. preparing a solution of sodium hydride (60% in oil, 8.4 g, 210 mmol, 1.8 eq. per OH group of sucrose) washed serially with dry hexanes (4×15 mL) in dimethylsulfoxide; cooling said solution to about 10° C.;

b. adding a solution of sucrose (5.0 g, 14.62 mmol, 116.8 mmol OH groups) in 30 mL dimethylsulfoxide;

c. heating to about 35-40° C. and stirring for about 90 minutes;

d. cooling a resulting mixture to about 10° C. and treating with methallyl chloride (13.6 g, 14.8 mL, 150.22 mmol, 1.3 eq. per OH group, added over 30 minutes), allowing same to attain a temperature of about 40° C., and then stirring overnight;

e. quenching said mixture with 5% aqueous sodium hydroxide (30 mL) at about 15° C., diluted with water (500 mL), and extracting with ethyl acetate (4×100 mL);

f. combining the resulting organic layers, washing serially with water, hydrogen peroxide (5% solution in water), water and brine (3×150 mL each), drying over anhydrous sodium sulfate, filtering through charcoal, then concentrating in vacuo.

In another alternate method of making epoxidized monomers, sorbitol (3.56 g, 19.43 mmol) is exchanged for said sucrose (5.0 g, 14.62 mmol,). In another alternate method of making epoxidized monomers, xylitol (3.55 g, 23.33 mmol) is exchanged for said sucrose (5.0 g, 14.62 mmol).

The methods of the present invention also include a method of making an epoxidized monomer disclosed above, comprising the steps of epoxidizing methallyl saccharide-based monomers with peracids to generate epoxymethallyl saccharides. One general method includes the steps of:

a. refrigerating a vessel charged with a methallyl saccharide-based monomer dissolved in ethyl acetate, and adding sodium acetate;

b. cooling said vessel, and adding peracetic acid dropwise;

c. heating said vessel to 10° C., and stirring said contents;

d. diluting said contents with ethyl acetate, and washing serially with cold water, cold aqueous saturated sodium carbonate and brine;

e. separating an organic layer, drying same, filtering same, and concentrating epoxymethallyl saccharide-based epoxy monomers.

Another method includes the steps of:

a. placing a vessel in a refrigeration bath, charging same with methallylsucrose dissolved in ethyl acetate, and adding sodium acetate in an amount equal to about 10% of the number of moles of peracetic acid to be added later;

b. cooling said vessel to about 5° C. and adding about 5.751 moles of peracetic acid dropwise over two hours;

c. heating to about 10° C., and stirring said contents overnight;

d. diluting said contents with ethyl acetate, transferring same to a separatory funnel and washing same serially with cold water, cold aqueous saturated sodium carbonate and brine;

e. separating an organic layer, drying same over anhydrous sodium carbonate, filtering same, and concentrating same in vacuo.

In another method of making epoxidized monomers, said saccharide-based monomer is sorbitol. In another method of making epoxidized monomers, wherein said saccharide-based monomer is xylitol.

The method of the present invention also includes method of making a polymerized epoxy mixture disclosed above, comprising the step of mixing saccharide-based monomers and a curing agent. Unless specified otherwise, curing, for this method and any other method disclosed herein, may be through heat and/or the passage of time.

The method of the present invention also includes a method of making an adhesive disclosed herein, comprising the step of mixing said saccharide-based epoxy monomers and said curing agent.

The method of the present invention also includes a method of making a coating disclosed herein, comprising the step of mixing said saccharide-based epoxy monomers and said curing agent and said viscosity modifier.

The method of the present invention also includes a method of making a composite disclosed herein, comprising the step of mixing said saccharide-based epoxy monomers and said curing agent and said filler.

To obtain a hard or load bearing composite, the method may include the steps of:

a. mixing said vegetable matter and concrete in water;

b. in a separate container, mixing said epoxy, curing agent and viscosity modifier;

c. mixing both mixture a. and mixture b. together; d. molding same to desired shape.

Although curing may be through the passage of time, one preferred manner of curing includes heat cure, first to about 80° C. until stiffening begins (to prevent running of the mixture), then heating to about 120° C. degrees until fully cured in the desired shape or configuration. To obtain composites that are more flexible, moldable, or that have insulative qualities, the method may include the steps of creating a webbing or mat by layering said filler, polyester and polypropylene, then applying a mixture of said saccharide-based epoxy monomers and curing agent. Before curing becomes established, it is preferred to configure said webbing mixture to the desired shape.

### Monomer Characterization

Differential scanning calorimetry (DSC) can be used to establish conditions for curing and to analyze the glass transition temperatures (Tg's) of cured thermosets. Thermogravimetry (or thermogravimetric analysis, TGA) is used to determine the decomposition temperatures of monomers and polymers and the char content remaining after degradation. Dynamic mechanical analysis (DMA) is used understand the viscoelastic behavior materials. Examples of such methods are set forth in Epoxy Resins by May (see, Tanaka et.al., in Epoxy Resins: Chemistry and Technology, 2^{nd} Edition, May, C. A. (editor), Marcel Dekker, New York, 1988, pp 285-463; Mika, T. F.; Bauer, R. S. idem, pp 465-550). The description of the preparation of epoxy resins and conditions therefore in these references are hereby incorporated by reference in their entirety.

The EMS monomers produced in accordance with the present invention may be characterized, for example, by chromatography, one-dimensional NMR techniques proton and carbon-13, and mass spectroscopy.

¹H NMR Spectrometry

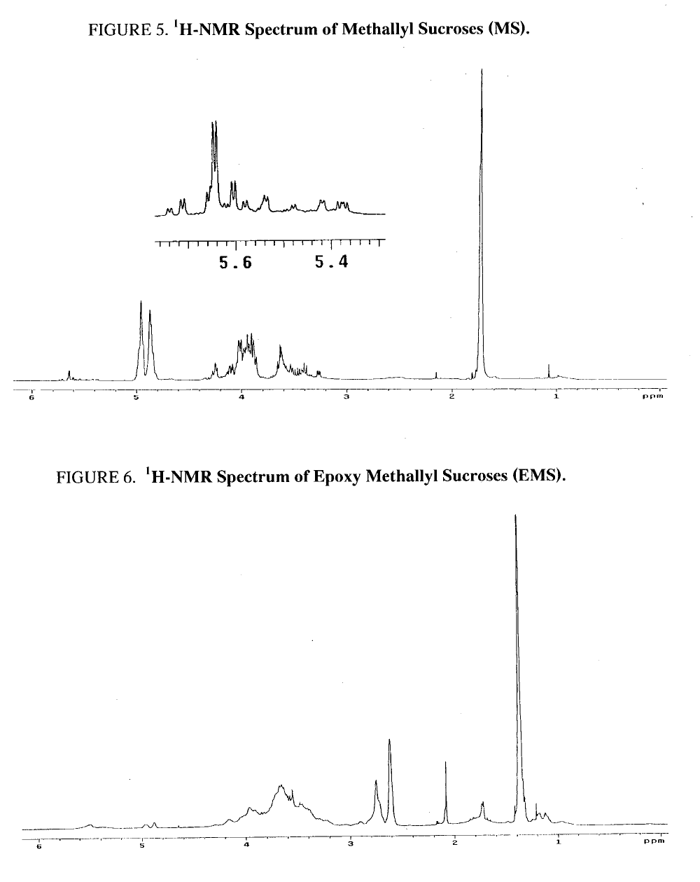

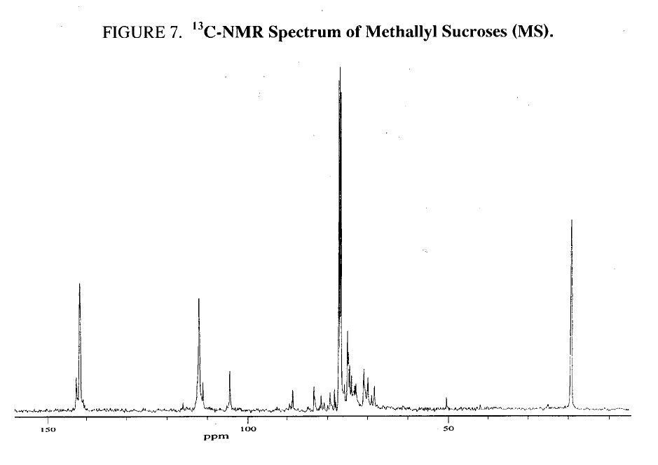

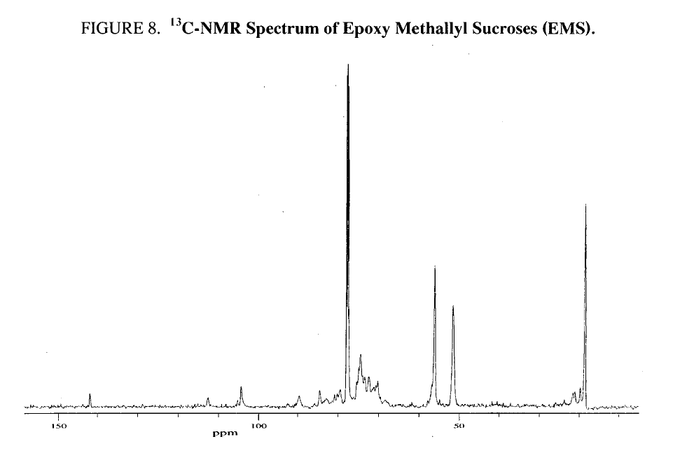

¹H-NMR for MS (CDCl₃) δ (ppm): 1.72 (allylic H-d, FIG. 5), 3.26-4.28 (sucrose[s] resonances and methylenes H-a), 4.82-4.98 (geminal terminal olefin hydrogens, H-c), 5.36-5.76 (15 doublets corresponding to the H-1 resonances of the glucopyranosyl moieties of the methallyl sucrose isomers). FIG. 6 is for EMS. ¹³C-NMR for MS (CDC₃) δ(ppm): 19.24 (allylic CH₃-d, FIG. 7), 68.23-83.40 (sucroses resonances and methylenes CH₂-a), 88.73 and 89.46 (C1 resonances of the glucopyranosyl moieties of the methallyl sucrose isomers), 104.13-104.44 (C2 resonances of the fructofuranosyl moieties of methallyl sucrose isomers), 110.95 B 112.87 (CH₂-c), 140.81 B 142.62 (tetrasubstituted olefin carbons C-b). FIG. 8 is for EMS.

¹H-NMR for EMS (CDCl₃) δ(ppm): 1.36 (H-d=, FIG. 6, epoxidized methallyl groups), 1.72 (allylic H-d, residual unepoxidized methallyl groups), 2.08 (residual acetic acid or acetate ester by attack on epoxy), 2.67 (H-c=, d, ²J_{HH}˜6 Hz, geminal methylene of the epoxy group) 3.0-4.3 (sucrose resonances and methylenes H-a and H-a′), 4.82-4.98 (residual geminal terminal olefin hydrogens, H-c), 5.5 (H-1 resonances of the glucopyranosyl moieties of the epoxy methallyl sucrose isomers).

¹³C-NMR for EMS (CDCl₃) δ(ppm): 17.83 (CH₃-d′ methyl of the epoxy methallyl group, FIG. 8), 19.24 (residual allylic CH₃-d,), 51.5 (terminal epoxy methallyl carbon CH2-c′), 56.1 (internal epoxy methallyl carbon C-b′) 66 B 86 (sucroses resonances and methylenes CH₂-a=and CH₂-a), 89.6 (C1 resonances of the glucopyranosyl moieties of the epoxy methallyl sucrose isomers), 104.2 (C2′ resonances of the fructofuranosyl moieties of epoxy methallyl sucrose isomers) and 105 (C2′ residual resonances of the fructofuranosyl moieties of unepoxidized methallyl sucrose isomers), 111.7 (residual CH₂-c), 141.3 (residual tetrasubstituted olefin carbons C-b).

Degree of Methallyl Substitution in MS Quantitative ¹³C-NMR: In the ¹³C spectra of MS (FIG. 7), the integral at ˜104 ppm belonging to quaternary fructofuranosyl carbon (C2′) was set at 100 integral units, and was used to compare the integrals of the terminal and internal olefin resonances at 111 and 141 ppm, respectively (678.3 and 693.3 integral units; average ˜685.8). The degree of methallyl substitution (DS) was calculated using the equation [DS=average of the integrals at 111 and 141 ppm divided by the integral at 104=(685.8 divided by 100˜6.8 methallyl groups per sucrose)]. Correspondingly, the average molecular weight of this mixture of monomers was (342.3-6.8+(6.8)55=709.5 g/mol). In this example 342.3=the molecular weight of sucrose; 6.8 the weight of protons lost upon methallyl substitutions, or the number of methallyl substituents; and 55=molecular weight of the methallyl fragment

Degree of Epoxy Substitution in EMS Quantitative ¹³C-NMR: The resonances at 51, 57, and 104 ppm in FIG. 8 correspond to the terminal and quaternary epoxy carbons and 2′-carbon of fructofuranose, respectively; and their integral values were 516.1, 598.6, and 100 units, respectively. The degree of epoxidation in EMS was determined to be 5.6 and was obtained by dividing the average of the integrals at 51 and 57 ppm (average=557) by the integral at 104 to 105 ppm (100 integral units). Correspondingly, the average molecular and epoxy equivalent weights of this mixture of epoxy methallyl monomers was 709.5+5.6(16)=799.1 g/mol. and 142.7 g, respectively. In this example 709.5 is the average molecular weight of methallyl sucrose; 5.6 is the average number of epoxy groups per sucrose; 16 is the atomic weight of oxygen; and the epoxy equivalent weight value 142.7 was obtained by dividing 799.1 by 5.6.

Mass Spectrometry

Fast atom bombardment (FAB) mass spectra were obtained on VG Instruments (Model 70 SE) using xenon as a bombarding gas. The molecular ions were detected as [M minus H+Na]⁺ for methallyl sucroses, and [M minus H+Na]⁺ and [M minus 2H+2Na]⁺⁺ for epoxy methallyl sucroses.

FAB Mass Spectral data on MS: For MS, molecular ions corresponding to [C₄₄H₇₀O₁₁+H]⁺, m/z=776.02 were expected. However, the distribution of molecular ions seen for MS corresponded to [C₄₄H₇₀O₁₁ minus H+Na]⁺, m/z=797; [C₄₄H₇₀O₁₁ minus H+Na minus C₄H₇]⁺, m/z (797 minus 54)=743; [C₄₄H₇₀O₁₁ minus 2H+Na minus 2C₄H₇]⁺, m/z (797 minus 108)=689; [C₄₄H₇₀O₁₁ minus 3H+Na minus 3C₄H₇]⁺, m/z (797 B 162)=635 amu.

FAB Mass Spectral data on EMS: For EMS we expected molecular ions corresponding to [C₄₄H₇₀O₁₉+H]⁺, m/z=904. However, molecular ions seen for EMS corresponded to [C₄₄H₇₀O₁₉+2Na]⁺, m/z=949; [C₄₄H₇₀O₁₉ minus H+Na]⁺, m/z (949 minus 23)=925; and a base peak at 856 corresponded to [C₄₄H₇₀O₁₆+H]⁺.

Densities

A 25 mL pycnometer and a constant temperature bath were used to measure the densities of the monomers from 25° C. to 50° C. (5° C. steps, Table 1). The pycnometer was filled with distilled water, allowed to equilibrate to a given temperature, and weighed. The pycnometer volume was determined by multiplying the weight of water by its density at each temperature (CRC Handbook of Chemistry and Physics, R. C. West and M. J. Aslte Editors, CRC Press Inc., Boca Raton, Fla., 1983, F-5). Monomers were then added to the pycnometer and their weights were measured as a function of temperature. Monomer densities at each temperature were determined by dividing the weight of the liquids by the volume of the pycnometer.

The mass and volume of the dry empty pycnometer were 21.171 g and 24.212 0.001 mL, respectively. Densities of the liquid monomers were determined by dividing the mass of each monomer (pycnometer plus monomer mass, minus pycnometer mass) by pycnometer volume at each temperature (Table 1). When these data were plotted as a function of temperature, they generated straight lines depicting the linear dependence of density with temperature (Table 1).

All three types of sucrose-based unepoxidized (MS, AS and CS) and epoxidized monomers (EMS, EAS and ECS) are liquids at room temperature. Densities for the unepoxidized monomers increased in the following order: CS<AS<MS. Densities for the epoxidized monomers increase in the order ECS<EMS<EAS. Epoxidation increased the masses and the densities of the monomers because double bonds were replaced with oxygen atoms.

Viscosities

Viscosities were measured as a function of temperature using calibrated and serialized Cannon-Fenske viscometer tubes (see the experimental on density). Flow times (seconds) for the meniscus of the fluid to pass between the two lines on the viscometer tubes were monitored with a stopwatch. Five determinations were made per monomer per temperature. Times were averaged and multiplied by the viscometer tube constant to yield the kinematic viscosity in centistokes (cS). The product of the kinematic viscosity and monomer density (Table 1) yielded dynamic viscosity in centipoise (cP, 1 cP=1 mPa-sec).

Table 2 shows the dynamic viscosities (flow under the influence of gravity) of the monomers. Viscosities (η) decreased asymptotically when plotted against temperature. The natural log (In η) vs. 1/T plots showed Arrhenius behavior over the range of temperatures investigated (Table 2).

The viscosities of the unepoxidized and epoxidized monomers increase in the order AS<CS<MS and ECS<EAS<EMS, respectively. The Arrhenius behavior of the viscosity of sucrose-based monomers [η(T)=ηₒ exp (E_{η}/RT)], was observed as shown in Table 2. The slopes of the regression lines yielded the flow activation energies (E_{η}, kJ/mol) and the y-intercepts gave In ηₒ. R, the universal gas constant in these plots, was taken to be 8.314 J/mol K. As shown in Table 2, flow activation energies (energy needed to induce flow) increased with increasing viscosities for each monomer.

Monomer Tgs BY DMA and DSC

Glass transition temperature (“Tg”) is the temperature at which the physical characteristics of some materials change from being glassy to becoming rubbery and soft. Tg can be a direct measure of the load bearing capacity of some polymers, within a certain temperature range, correlating with the relative stiffness (modulus) of the polymer.

The Tgs of the sucrose-based derivatives were determined on a Perkin-Elmer dynamic mechanical analyzer (DMA-7e) in the static mode using a 3 mm sphere probe from −100° C. to 25° C. (2° C./min). Samples were analyzed in open aluminum DSC pans, and probe position was monitored as a function of temperature. Two intersecting tangents continuing from the slopes of the probe position curve were drawn, and the intersection was defined as the onset of the Tg (Table 3). Tgs by DSC were obtained using a Shimadzu DSC 50 and were used to correlate those obtained by DMA. DSC Tgs were obtained from the midpoint between the initial and shifted baselines. All thermal studies (DMA & DSC) were conducted in a nitrogen atmosphere (flow rate=20 mL/min).

Table 3 shows the values for Tgs for the sucrose-based monomers. AS, CS and MS exhibited Tgs (by DMA) of −78.3° C., −70.7° C. and −41.9° C., respectively; the epoxy monomers (EAS, ECS and EMS) showed Tgs of −46.3° C., −25.6° C. and −22.8° C., respectively. The data for the unepoxidized monomers (AS, CS and MS) appeared to show an increase in Tg with the increase in size (and perhaps branching) of the ether appendage. This was also observed for the three epoxy monomers (EAS, ECS and EMS). In comparison, DGEBA is a solid at room temperature and melts between 41° C. and 44° C. (Aldrich ref). DSC was also used to investigate the softening points (Tgs obtained by DMA). DMA and DSC softening points (Tgs) appear to be identical. Therefore, sucrose-based monomers are amorphous solids at low temperature since their DSC curves showed glass transition type discontinuous changes in heat capacity behavior.

Biological: Modified Maron-Ames Tests

EAS, EMS, ECS and DGEBA (100 μL each) were diluted in 900 μL dimethylsulfoxide (DMSO, 10⁻¹ dilution). Serial dilutions (100 fold) were made with DMSO to obtain samples with dilution factors of 10⁻³, 10⁻⁵, and 10⁻⁷. The assays were performed according to the modified methods of Maron and Ames (Maron D. R. and Ames, B. N. Mutation Research, 1980, Vol. 113, 173-215) using two strains of Salmonella Typhimurium TA-98 and TA-100, with and without metabolic activation of the substrate. The male rat microsomal homogenate S-9 was used to effect metabolic activation of the substrate. TA-98 and TA-100 were cultured in Oxoid Nutrient Broth No.2 at 37° C. for 16 hours using a gyratory shaker (200-250 rpm). Strains were tested to confirm viability of culture, membrane permeability, and the integrity of genetic markers using the following methods: (1) Histidine Requirement: A positive result of this test (growth in histidine/biotin plates; no growth in non-histidine/biotin plates) showed that the histidine/biotin-dependent mutants were present. (2) Crystal Violet: Results of this test showed that bacterial membranes were permeable. (3) Ampicillin Resistance (R factor): Tester strains showed growth on ampicillin plates indicating that the Salmonella strains were ampicillin resistant and bore its marker.

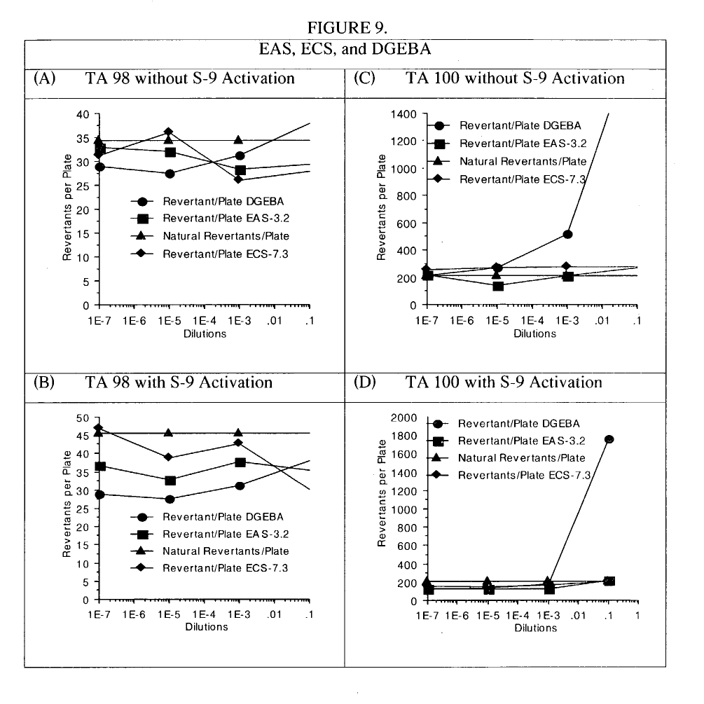

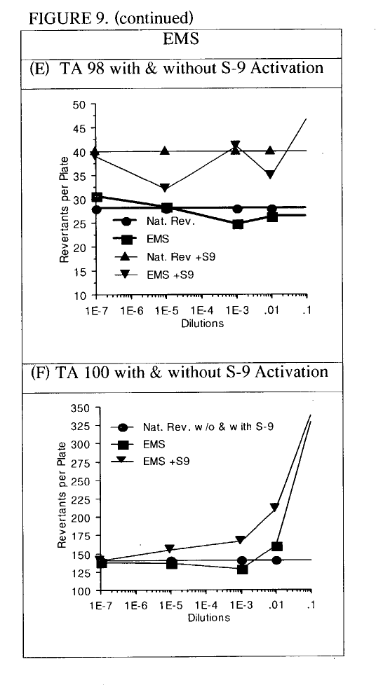

Compounds were assayed after the bacteria tested positive for the viability-related criteria above. Two mL top agar was placed in sterile culture tubes and kept at ˜40° C. in a water bath. Samples (100 μL) were placed in each tube (3 tubes per sample) and 100 μL of either strain TA-98 or TA-100 was added. When required, the S-9 homogenate (500 μL) was then added. The tubes were mixed and poured into minimal glucose agar plates. The plates were left at room temperature until the top agar solidified. Then, they were inverted and incubated at 37° C. for 48 hours. After incubation, revertant colonies were counted. Samples that presented greater than 2 times the number of revertant colonies were considered mutagenic. (See FIGS. 9(a) through (f)).

Salmonella Typhimiurium strains TA-98 and TA-100 were deemed useful, based on the criteria stated above. FIGS. 9(a) through (f) depict the cytotoxicity and mutagenicity profiles of EAS, ECS, EMS and DGEBA. Figures (a) and (b) show that there was no apparent mutagenicity from any of the four epoxies tested using Salmonella Typhimiurium strain TA-98 (with or without metabolic activation S-9). Although the four compounds appeared to be slightly cytotoxic with microsomal enzyme S-9 activation, there was no effect on the background lawn. That is, the number of revertant colonies for all concentrations remained about the same. Thus, these compounds were deemed to be neither cytotoxic nor mutagenic to tester strain TA-98 under the experimental conditions.

Using Salmonella Typhimiurium TA-100, no mutagenic potential was observed with either EAS or ECS. However, there was a mutagenic response observed in samples containing DGEBA at dilutions greater than 10⁻⁵ without S-9 activation (Figure (c)), and at dilutions greater than 10⁻³ with S-9 activation (Figure (d)).

Using Salmonella Typhimiurium TA-98, EMS showed no mutagenic response with or without S-9 activation (Figures (e)). However, a mutagenic response was observed with EMS using strain TA-100 at dilution factor 10⁻¹ (100 ML/900 ML DMSO), with or WITHOUT S-9 activation (Figure (f)). With EMS, at dilution factor 10⁻², the revertant colonies had not doubled. Therefore, EMS may be considered borderline cytotoxic at dilution factors of 10⁻¹. At dilution factors beyond 10⁻¹, EMS was neither cytotoxic nor mutagenic under these experimental conditions.

DSC Curing Studies

Peak curing temperature is an indication of the temperature at which maximum combination of reactants occurs to produce a thermoset (cross-linked polymer that will not be re-formable upon heating). The lower the peak curing temperature, the faster the epoxy will react with the curing agent (hardener) to form a thermoset. Typically, the more efficient the epoxy is in its curing behavior, the less cost of energy will be required in processing the epoxy. Many types of applications require epoxy having curing temperatures at or below ambient temperature; one example is quick curing home repair kits. Other types of applications are better served by requiring higher curing temperatures. Peak curing temperature is measured by a differential scanning calorimeter (“DSC”), indicating the temperature at which maximum exotherm occurs.

Curing reactions were performed on a Shimadzu DSC-50 and the instrument was calibrated using indium and tin. Sucrose-based epoxidized monomers were mixed with the amines in ratios as shown in Table 4. Five different curing conditions were studied for each monomer. To minimize reaction between the epoxy and the amine before curing could be studied, the epoxy amine formulations were rapidly combined, mixed, placed in open aluminum pans and transferred to a pre-chilled sample chamber (<2 min). The materials were then heated from −25° C. to +250° C., at 5° C./min. Cure temperatures were obtained from the peak temperature of the curing curves, and heats of cure (ΔH, cal/g) were calculated from the area under the curves using software provided by Shimadzu (Table 5). These were then converted to enthalpy values, ΔH (kJ/mol epoxy group) using the formula ΔH (kJ/mol epoxy group)=[experimental heat of cure (cal/g)]×[(weight of epoxy+amine, mg)÷weight of epoxy, mg)]×[molecular weight of epoxy (g/mol)]×[1 mol epoxy÷N]×[1 kcal/1000 cal]×[4.184 KJ/1 kcal], where N is the average number if mol of epoxy groups per sucrose as determined by ¹³C-NMR (above). The extent of reaction (% cure) was obtained from the ratio of the calculated ΔH and the theoretical ΔH of cure (=119.19 kJ/mol). Bond energy values for the theoretical heat of cure per mole epoxy were obtained from the text of Vellacio and Kemp (Vellacio, F., and Kemp, S. Organic Chemistry, Worth Publishers, Inc., N.Y. 1980, p. 1058) Using the formulations in Table 4, EAS, EMS and ECS were individually cured with DETA, UNI-REZ 2142 (polyamide) and UNI-REZ 2355 (polyamidoamine), to obtain information on their curing behavior in comparison to DGEBA. Table 5 shows peak curing temperatures, heats of cure (kJ/mol), extents of cure (% cure), and Tgs for the thermoset formulations. The peak curing temperatures and heats of reactions were determined during the initial heating scans from the large exotherms observed. Subsequent heating scans showed no exotherms, and reactions were deemed complete after the initial heating cycle. Thereafter, Tg data were obtained on the thermosets after two programmed heating and cooling cycles to enable the polymers to relax naturally. Data are averages of four samples.

EAS cured readily with DETA. When the epoxy: NH ratio was 1:1, the average peak curing temperature and Tg were ˜97.8° C. and 32° C., respectively. With UNI-REZ 2142 (40 and 70 phr) average peak curing temperatures, heats and extents of cure, and Tgs increased with increasing amine concentrations. The same effects were also observed for EAS at both concentrations of UNI-REZ 2355.

EMS readily cured with DETA. When the epoxy: NH concentration was 1:1; the average peak curing temperature, and heat and extent of cure were 75.5° C., 76.6 kJ/mol, and 64%, respectively. EMS thermosets did not provide DSC observable Tgs (however, Tgs for these thermosets were obtained by DMA, see below). Average peak curing temperatures of the mixtures containing EMS and UNI-REZ 2142 (at 40 and 70 phr) were 99.6 and 88.1° C., respectively; the heats and extents of cure were 72.0 kJ/mol and 60.1% (for 40 phr) and 107.2 kJ/mol and 89.5% (for 70 phr), respectively. As amine concentration increased the average curing temperatures decreased and the heats and extents of cure increased. The same effects were observed when EMS was cured with UNI-REZ 2355 at 40 and 70 phr.

With ECS, increasing concentrations of DETA were needed to observe adequate cure. As the ratio epoxy: NH, increased from 1:1 to 1:3, more ECS was observed to react. Concomitantly, the average heats and extents of cure increased. With increasing concentrations of both UNI-REZ 2142 and UNI-REZ 2355 (40 and 70 phr), ECS showed increases in peak curing temperatures, and heats and extents of cure. The significant difference between UNI-REZ 2142 and UNI-REZ 2355 was that ECS cured at higher curing temperatures with UNI-REZ 2142 and at lower temperatures with UNI-REZ 2355. The Tgs for the two ECS samples could not be clearly observed by DSC.

When DGEBA was cured with DETA (epoxy: NH, 1:1), the peak curing temperature was 96.9° C., and the heat and extent of cure were 103.8 kJ/mol, and 86.7% respectively. With UNI-REZ 2142 cure occurred at 107.1° C. (40 phr) and 105.2° C. (70 phr), respectively. Heats and extents of cure increased with increase in the concentration of 2142. This was also observed when DGEBA was cured with 40 and 70 phr UNI-REZ 2355. DSC was not suitable for obtaining the Tgs of these thermosets.

The theoretical heat of cure for reactions involving epoxy groups and amines, as determined by Hess's law using bond energy and ring strain values, is 119.75 kJ/mole epoxy group (Vellacio & Kemp). Extents of reactions were determined by dividing the experimental ΔH (kJ/mol) by the theoretical heat of cure (119.75 kJ/mol, Sachinvala et al. Journal of Polymer Science, 1998, vol. 36, 2397-2413).

ΔH values for the DETA cured EAS, EMS, ECS and DGEBA thermosets were 87.9, 76.6, 32.2 and 103.8 kJ/mol, respectively (Table 5). Here the epoxy: NH ratio was 1:1. The ΔH values for the curing of DGEBA with DETA approached the theoretical heat of cure, indicating near complete reaction (86.7%). The lower extents of reactions observed for EAS and EMS (73.4% and 64%) may be attributed to the hindrance afforded by neighboring allyl and methallyl groups to the approaching amine nucleophiles. In addition to the examples shown here, EMS was found to be a very reactive monomer, it reacted with amines, polyamines, thiols, polythiols, amino acids, anhydrides (in presence of tertiary amines and phosphines) and the like. Viscosities increased dramatically while mixing EMS with DETA at room temperature as well as at 0° C. Therefore, the experimentally determined heat of cure may not be a true representation of the extent of cure, since attempts to mix the two reagents at 0° C. did not improve the quality of our data.

The significantly lower extent of reaction for DETA and ECS (26.9%) can be attributed to the curing behavior of internal epoxy groups. In these systems (internal epoxy groups), only primary amines (RNH₂) react, and the amine-to-epoxide reaction stops when a secondary amine is formed (i.e. when one hydrogen from RNH₂ is consumed Sachinvala, et al. J. Polymer Science, 1998, vol. 36, 2397-2413.) This argument is supported by the fact that when ECS was reacted with benzyl amine at 150° C., all epoxy groups had reacted and no epoxy peaks were observed at 51 ppm by C-13 NMR. However, the resulting benzyl amine adduct was completely soluble. This indicated that no thermoset formation had occurred, and the reaction stopped when only one NH was consumed. The higher temperatures needed to cure ECS (˜150°), epoxies are known to cure significantly by etherification (Sachinvala, et al., Journal of Polymer Science 1998, vol. 36, 2397-2413) and only release ring strain energy contributes to the theoretical ΔH value of 100.1 kJ/mol. Therefore, the extent of cure could be higher than what is reported from the curing data. While increasing the epoxy: NH ratio from 1:1 to 1:2 and 1:3 increased the extent of reaction (50.7% and 59.1%), however, the extra amine also cause blushing problems and lowered glass transition temperatures.

Based on % cure with DETA, the order of the reactivities of the four epoxy systems is: DGEBA>EAS>EMS>ECS. However with DETA, peak curing temperatures of the four epoxies increase the order EMS<<DGEBA and EAS<<ECS.

UNI-REZ 2142 reacted readily with EAS, EMS and DGEBA at 40 phr, and showed heats of cure (and extent of reaction) of 65.3 kJ/mol (54.5%), 72.0 kJ/mol (60.1%) and 74.9 kJ/mol (62.2%), respectively. Increasing the mixing ratio of UNI-REZ 2142 to 70 phr raised the AH and extent of reaction (Table 5). The extents of reactions of UNI-REZ 2142 with DGEBA and EMS at 40 and 70 phr were greater than those with EAS at the same mixing ratios. With ECS, which contains internal epoxy groups, UNI-REZ 2142 reacted less readily at both concentrations, as was evidenced by the much lower heats and extents of cure.

An interesting phenomenon was observed in the curing of the epoxies with 2142. With epoxies that cure at lower temperatures (EMS and DGEBA), a decrease in the peak curing temperature was observed as the concentration increased. This is because, with increasing UNI-REZ 2142 concentration, more amine was available to combine with the faster acting epoxy groups, and networks formed at lower temperatures gel point reached quicker and at lower temperatures while also showing increased extent of reaction. EMS prepared in aqueous solutions is a faster reacting epoxy system because there are hydroxyl groups present on sucrose as a consequence of incomplete methallylation in aqueous sodium hydroxide. And hydroxyl groups are known to catalyze the curing of epoxies at lower temperature (Sachinvala, et al., J. Polymer Science, 1998, vol. 36, 2397-2413). DGEBA is also a faster reacting epoxy pre-polymer. It too has hydroxyl groups present in the pre-polymer because the reaction of bisphenol-A and glycidyl chloride forming glycidyl end-capped polyether polyols. However, unlike EMS, it does not cure readily below room temperature, and at 0° C, DGEBA and DETA mixtures are relatively unreactive.

When EAS and ECS were cured, the peak curing temperatures increased with amine concentration. This effect was very pronounced in the curing of EAS. The peak curing temperatures were 92.5° C. (40 phr) and 107.3° C. (70 phr). This may be because the EEW is 223 and the reactivity of its epoxy groups are somewhat hindered by the neighboring allyl groups. As EAS reacted with UNI-REZ 2142 and networks emerged in the system, the viscosity increased and higher temperatures were needed (to surpass viscosity barriers and gel formation) for the remaining amine groups to react. At gelatin, reaction kinetics change from concentration to diffusion controlled. Less pronounced increases in peak curing temperatures were noted for the reaction of UNI-REZ 2142 with ECS. This was because the curing temperatures of ECS and UNI-REZ 2142 were high enough (about 192° C.) to transcend the viscosity and gelation barriers. Furthermore, ECS has more epoxy groups available in the monomer to continue its reactions. That is why mixtures of ECS and amines (and polyamidoamines) are fluids during the course of their reactions, prior to gelation.

UNI-REZ 2355 reacted more completely at 40 phr with DGEBA than either EAS or EMS, as reflected by the higher heats of cure (and extents of reaction) 100.1 kcal/mol (83.6%), and 65.3 kcal/mol (54.5%) and 72.0 kcal/mol (60.1%), respectively. Increasing the amine concentration increased the heats of cure and extent of reaction for the four epoxies, but lowered Tgs in the cured thermosets (Table 5).

Glass Transition (Tg) by DSC

Cured samples were placed in the DSC at room temperature and heated to 200° C., at the rate of 10° C./min. The temperature was then held at 200° C. for 5 minutes, to allow stresses in the samples to relax. Subsequently, the samples were cooled to −100° C., and reheated to 200° C. at the rate of 5° C./min. This heating program was repeated three more times (total of four observations). Tgs were measured during the third and fourth heating runs. Tg values were determined from the forward step curve at the midpoint between the initial and shifted baselines.

Tgs reported in Tables 5 and 6 were obtained on the cured thermosets by DSC and DMA.

Dynamic Mechanical Analyses (DMA)

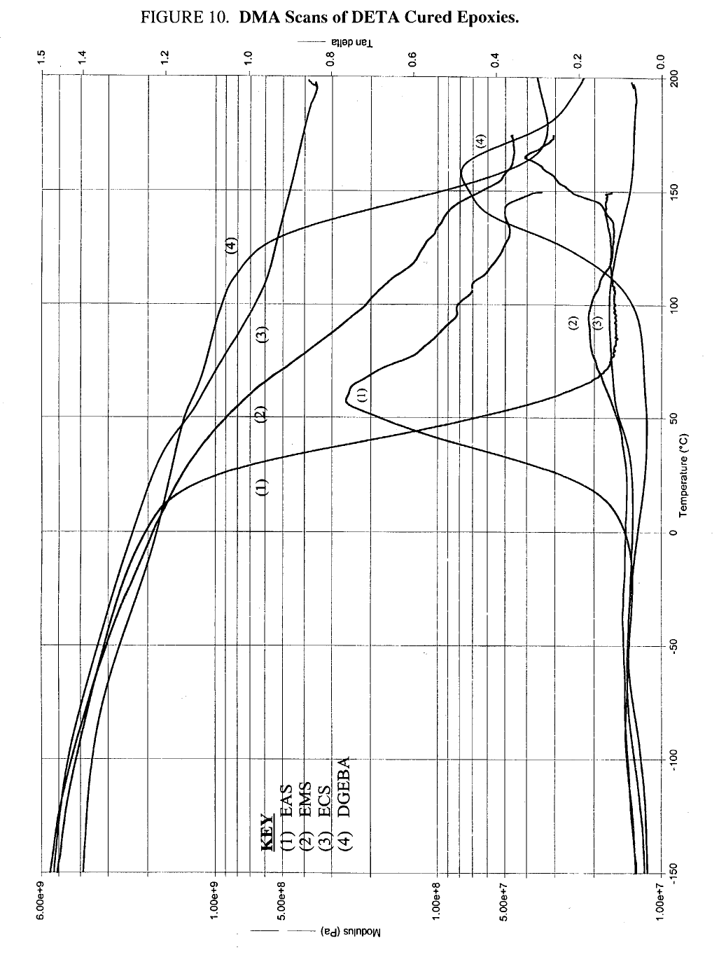

Samples for DMA analysis were prepared by mixing the epoxies with the amines (Table 4). The solutions were poured into Teflon® coated muffin pans and cured overnight (˜16 hours) at their peak curing temperatures (determined by DSC, Table 5). The materials were then slowly cooled to room temperature (over 2 hours), and cut into rectangular strips of average size: length (1)×width (w)×thickness (t)=20×7×1.5 mm. Their moduli and glass transition temperatures (Tgs) were observed by three point bending experiments in a Perkin Elmer DMA-7E from −150° C. to +200° C. at a heating rate of 5° C./min. The displacement amplitude was 10 μm, at 3 Hz, and the static force was set to 110%. Moduli are reported from the real parts (E′) of their complex dynamic moduli (E*) at 20° C. (Table 6). Tgs in the same table were ascertained from the peak temperatures of the loss moduli (E″, not shown in FIGS. 10, 11, and 12).

Cured thermosets were evaluated by three point bending mode (DMA) to determine the changes in the storage modulus E″ and tan δ as the temperature was varied from minus 150° C. to plus 200° C. These data are presented in FIGS. 10 through 12, and summarized in Table 6.

FIG. 10 shows the storage moduli (E′, log scale) and tan δ (E″/E′) plots for the four epoxies cured with DETA. For EAS, EMS, ECS and DGEBA thermosets, storage moduli (E′) at 20° C. appeared to be −1.1 GPa, 1.4 GPa, 1.8 GPa and 1.4 GPa. These values are the averages of 4 samples per thermoset. Below Tg, all materials (curves 1-4) showed a near linear decrease in modulus with increase in temperature. The materials experienced Tgs at ˜23°, 350, 50°, and 122° C., respectively, as determined from the peaks of their loss moduli (E″ not shown in FIG. 10, but the Tg values are recorded in Table 6). EAS and DGEBA thermosets (curves 1 and 4, FIG. 10) traversed Tg over a temperature range of ˜65° C., and showed ˜2.0 and ˜1.6 order of magnitude decrease in the elastic modulus E′ (during Tg). EMS thermosets experienced Tg over 100° C. range and showed a ˜1.5 order of magnitude decrease in elastic modulus E′ (curve 2). ECS thermosets showed only a slight inflection at 50° C. (curve 3); and no sharp drops in the elastic (storage) moduli were seen for ECS samples over the temperature range studied. That is, ECS samples showed only one order of magnitude decrease in E′ from minus 150° C. to +200° C.). Data for tan δ (E″/E′) for EAS and DGEBA (curves 1 and 4) showed large peaks cresting at ˜55 and ˜155° C., respectively. The area under EAS tan δ was larger than that under DGEBA tans curve. Indicating that EAS thermosets are weakly crosslinked. EMS and ECS thermosets showed broad and shallow tans (curves 2 and 3) indicating high crosslinking density.

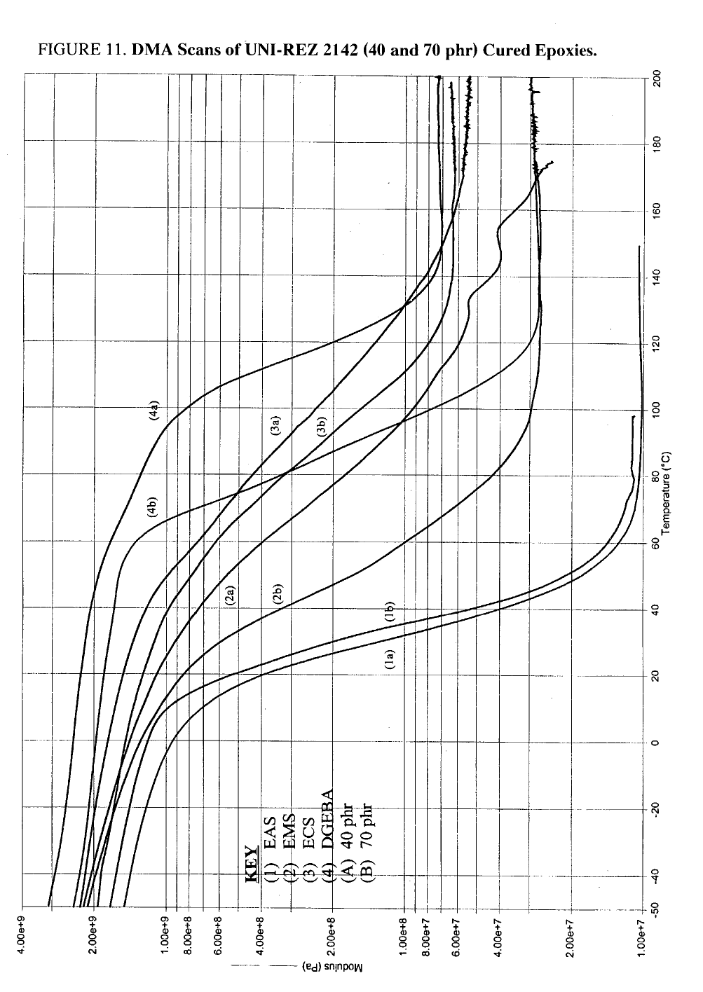

FIG. 11, shows eight E′ plots for the four epoxies cured with UNI-REZ 2142 at 40 and 70 phr (curves a and b, respectively) and shows the effects of increasing concentration of UNI-REZ 2142 on the E′ values and Tgs. Tan δ curves are not shown in this figure because the trends observed for the DETA cured thermoset repeated with these samples. For EAS (curves 1a and 1b), increasing the amine concentration increased E′. The range over which Tg occurred, and the extent of decrease in modulus (E′) during Tg for both curves was the same. For EMS (curves 2a and 2b), increasing the amine concentration decreased E′at 20° C., and Tgs occurred over a 100-degree range. The ECS elastic modulus (E′) curves (3a and 3b) are similar in shape and magnitude to each other. The higher amine concentration lowered E′ and shifted the 20 degree C. modulus to lower values. For the two DGEBA curves (4a and 4b) increase in the concentration of UNI-REZ 2142 characteristically decreased E′, Tg, and the modulus of the rubbery plateau.

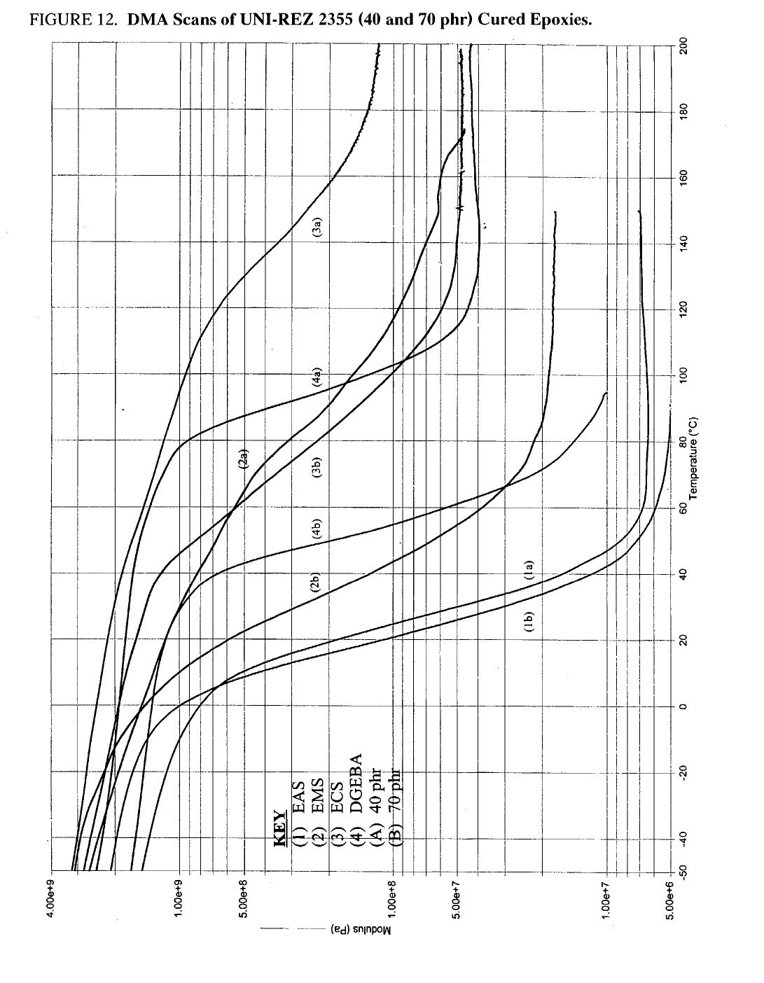

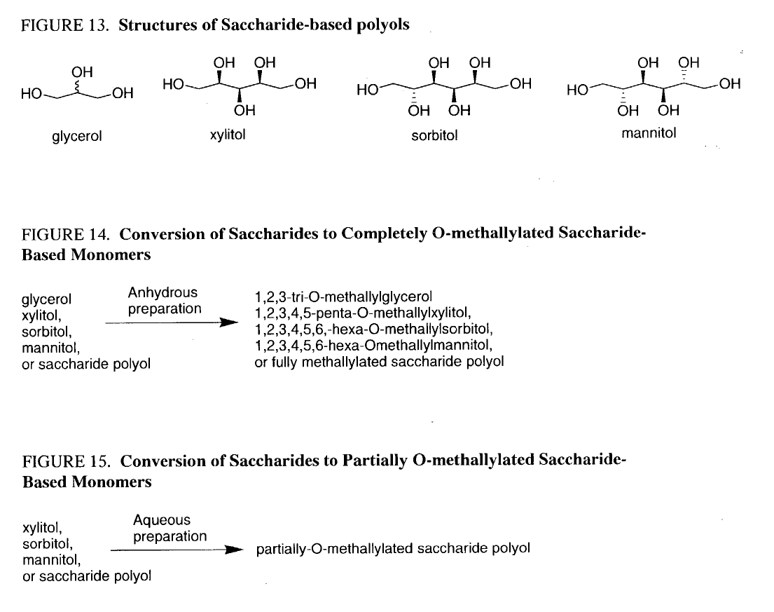

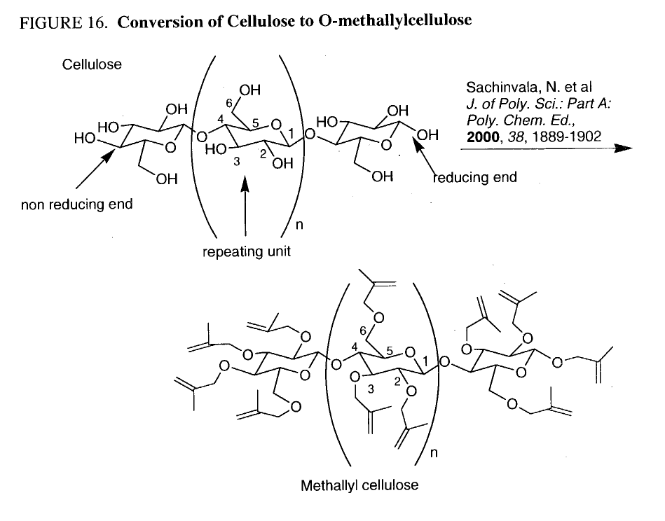

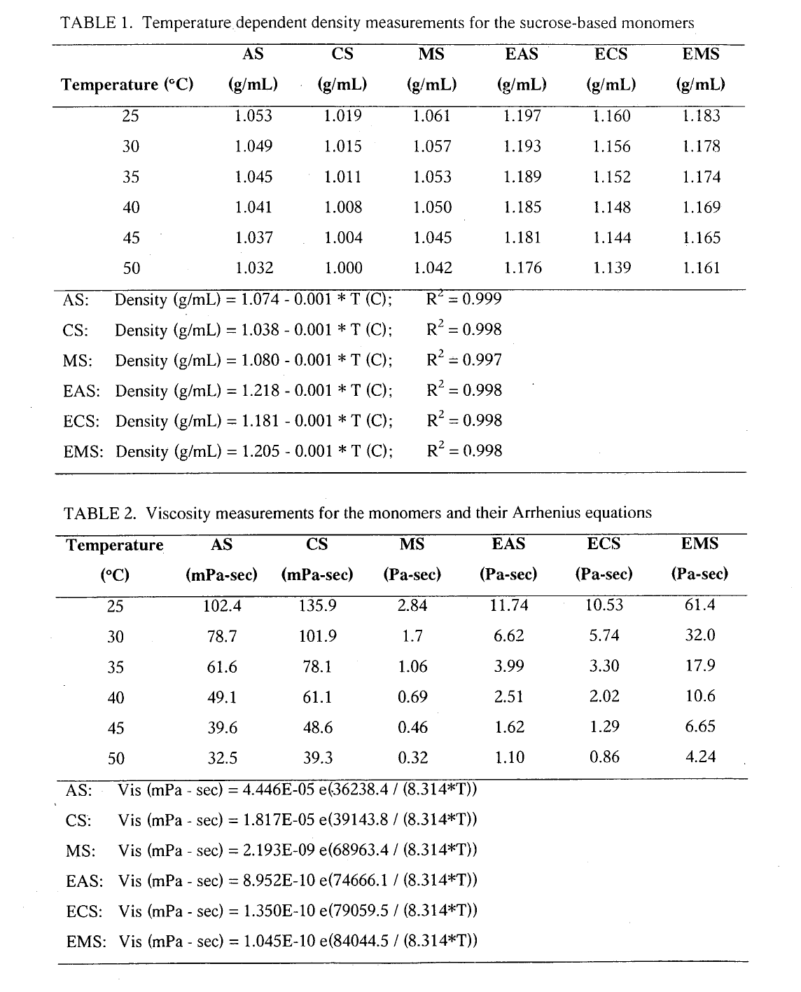

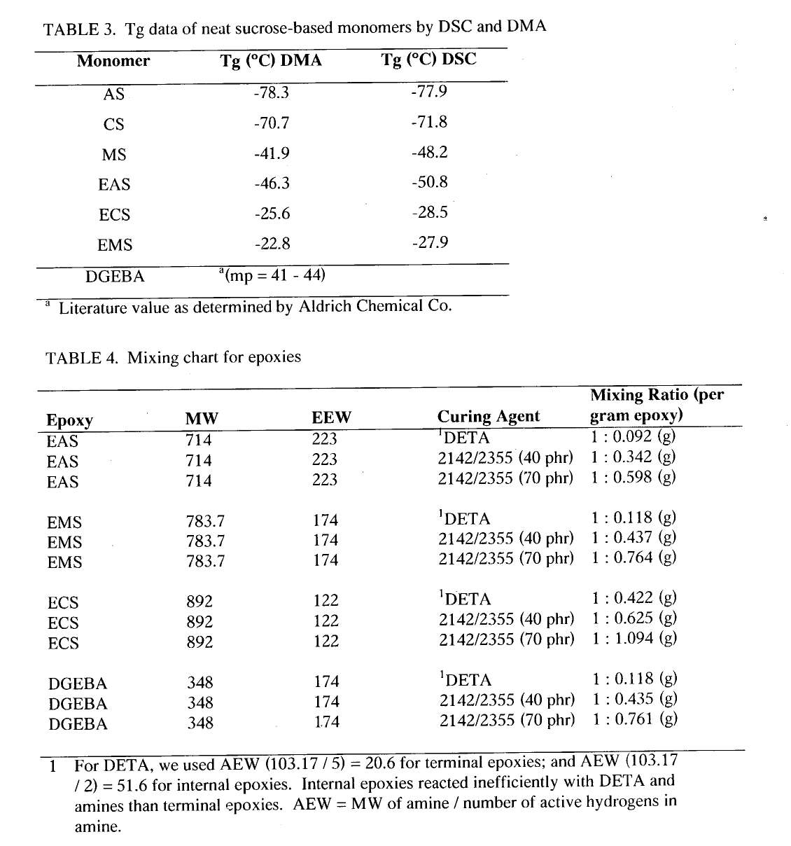

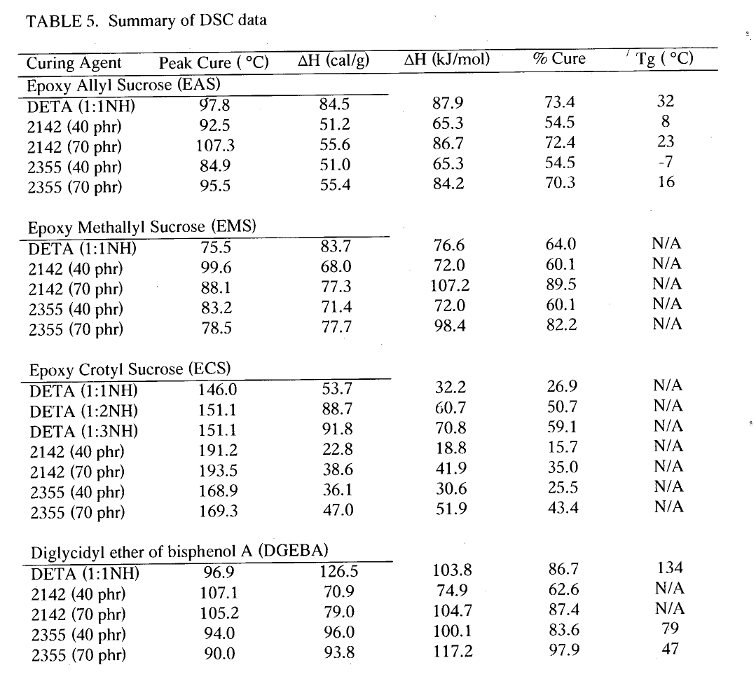

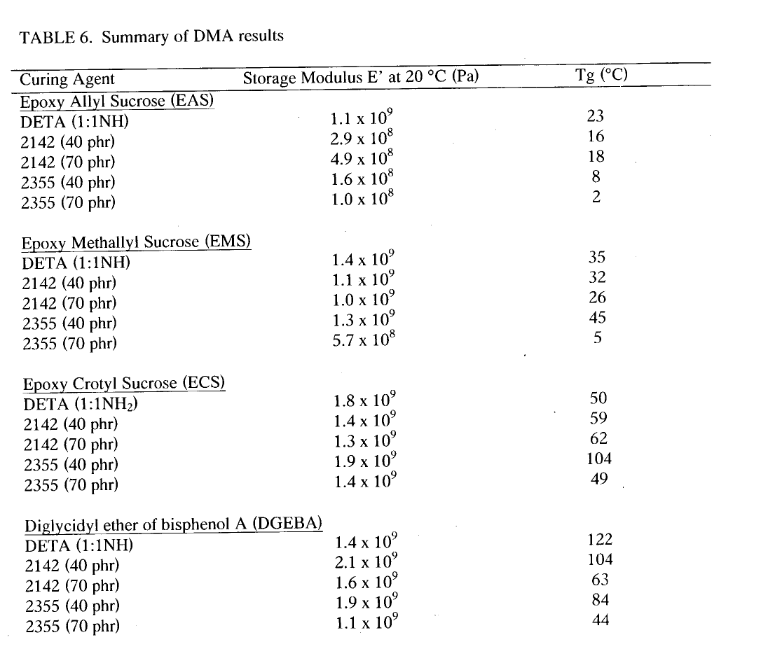

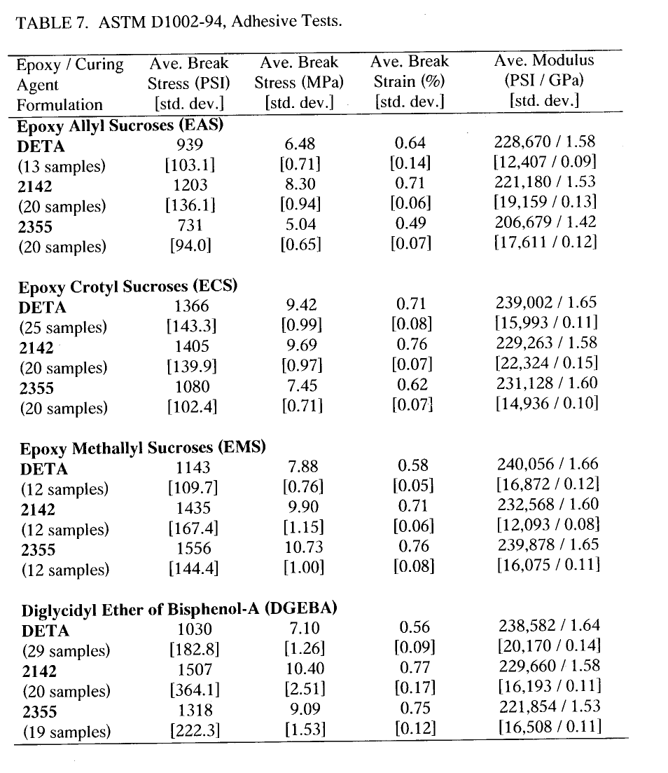

With UNI-REZ 2355 (FIG. 12), moduli and Tgs decreased with increase in amine concentrations (Table 6). The trends observed for UNI-REZ 2355 cured thermosets were similar to those observed for the thermosets cured with UNI-REZ 2142 (compare the curves in FIGS. 11 and 12). However, the differences in all of the properties for the two concentrations of UNI-REZ 2355 were more pronounced than those observed with 2142. ECS and UNI-REZ 2355 at 40 phr showed the highest modulus, Tg, and operating range for the entire set of epoxies studied with this curing agent.

Thermomechanical studies by DMA: EAS thermosets were flexible at room temperature. For the DETA cured thermoset, this material showed higher modulus and Tg because the amine equivalent weight (AEW) was significantly lower than that of either UNI-REZ 2142 or UNI-REZ 2355. This resulted in low molecular weights between crosslinks, higher crosslinking density, and a higher strength material. UNI-REZ 2142 and UNI-REZ 2355 are dimer acid derived amine curing agents. The reagents have high overall molecular weights, and molecular weights between reactive functional groups are also high. These aliphatic portions of UNI-REZ 2142 and UNI-REZ 2355 do not crosslink and plasticize the thermoset. However, with both UNI-REZ 2142 and UNI-REZ 2355, increasing concentration did improve the extent of reaction. (Table 5).

DGEBA showed a similar set of trends when compared with EAS (FIGS. 10 to 12, Table 6). All formulations showed a significant decrease in moduli (>2 orders of magnitude) as they traversed the Tg region (between 65° to 75° C.). In contrast to EAS, the DGEBA formulations were stiffer at room temperature (1.1 to 2.1 GPa). Increasing the concentration of UNI-REZ 2142 and UNI-REZ 2355 resulted in decreasing in both modulus and Tg because UNI-REZ 2142 and UNI-REZ 2355 acted as plasticizers, despite increased extent of reaction (Table 5).

When ECS was cured with DETA (FIG. 10, curve 3), the thermoset showed a near linear decrease in modulus over the entire temperature range examined, and a slight inflection in modulus at 50° C. (corresponding to Tg). The decrease in modulus of the ECS/DETA thermoset as it traversed glass transition was only one order of magnitude. This was the lowest of any of the epoxy/amine formulation studied. Furthermore, the ECS/2355/40 phr thermoset had the highest Tg of the ECS/amine formulations and also exhibited slight reduction in modulus. The remaining ECS thermosets showed similar trends wherein moduli decreased slightly during Tg over a 100-degree range.

EMS moduli and Tgs were generally higher than EAS, but lower that ECS, and moduli decreased gradually as Tgs occurred over a longer range of temperatures. This suggested that mechanical properties of EMS and ECS thermosets are unlikely to change as abruptly.

Adhesion Studies by Lap Shear

Aluminum strips (1×w×t, 4×1×0.044 inches) were cut and sanded (coarse sand paper 36 grit, using a Craftsman® grinder); and then washed serially with soap and water; deionized water; and acetone. The stripes were then dried in a vacuum oven (120° C., 1 hour, 0.1 mm Hg), cooled to room temperature in vacuo, opened under nitrogen, and coated and assembled immediately. No other surface preparation method was employed. Lap shear sandwiches (half inch overlap) containing the adhesive formulations (Table 4) were assembled using ACCO'M binder clips and cured (see DMA conditions). Bent samples were discarded and the strips were tested in an Instron® (Model 4201) according to ASTM D1002-94 (1000 lb. load cell, strain rate 0.05 in/min, Table 7).

Moduli (as determined from the slopes of the stress strain curves) for all samples ranged from 206,679 to 239,002 PSI (1.43 to 1.67 GPa). Adhesion strength trends for EAS, ECS, EMS, and DGEBA with the three curing agents DETA, and UNI-REZ 2355 and UNI-REZ 2142 may be expressed as follows:

With DETA: ECS>EMS>DGEBA>EAS

With UNI-REZ 2142: DGEBA>EMS≈ECS>EAS

With UNI-REZ 2355: EMS>DGEBA>ECS>EAS.

We claim:

1. A composition of matter, comprising an unepoxidized monomer comprising a non-sucrose saccharide-based molecule comprising at least one methallyl-containing group bonded to at least one hydroxyl group thereof.

2. The unepoxidized monomer of claim 1, wherein said bond is between said methallyl-containing group and at least one primary or secondary hydroxyl group.

3. The unepoxidized monomer of claim 1, wherein said methallyl-containing group comprises a long chain methallyl-containing ether group on said hydroxyl group.

4. The unepoxidized monomer of claim 3, wherein said long chain methallyl-containing ether comprises more than one double bond in the carbon chain.

5. The unepoxidized monomer of claim 1, wherein said non-sucrose saccharide-based molecule is fully substituted with methallyl-containing groups, each bonded to at least one different hydroxyl group thereof.

6. The unepoxidized monomer of claim 5, wherein said monomer is 1,2,3-tri-O-methallyl glycerol.

7. The unepoxidized monomer of claim 5, wherein said monomer is 1,2,3,4,5-penta-O-methallyl xylitol.

8. The unepoxidized monomer of claim 5, wherein said monomer is 1,2,3,4,5,6-hexa-O-methallyl sorbitol.

9. The unepoxidized monomer of claim 5, wherein said monomer is 1,2,3,4,5,6-hexa-O-methallyl mannitol.

10. The unepoxidized monomer of claim 5, wherein said monomer is a cellulose derivative wherein each of its hydroxyl groups are substituted with at least one methallyl-containing group.

11. A composition of matter, comprising a saccharide-based epoxy monomer comprising at least one geminally disubstituted terminal epoxy group per saccharide and at lease one geminally disubstituted terminal double bond.

12. A saccharide-based epoxy monomer of claim 11, wherein said epoxy comprises epoxymethallyl glycerol.

13. A saccharide-based epoxy monomer of claim 11, wherein said epoxy comprises 1,2,3 tri-O-epoxy methallyl glycerol.

14. A saccharide-based epoxy monomer of claim 11, wherein said epoxy comprises epoxymethallyl xylitol.

15. A saccharide-based epoxy monomer of claim 11, wherein said epoxy comprises 1,2,3,4,5-penta-O-epoxymethallyl xylitol.

16. A saccharide-based epoxy monomer of claim 11, wherein said epoxy comprises epoxymethallyl sorbitol.

17. A saccharide-based epoxy monomer of claim 11, wherein said epoxy comprises 1,2,3,4,5,6-hexa-O-epoxymethallyl sorbitol.

18. A saccharide-based epoxy monomer of claim 11, wherein said epoxy comprises epoxymethallyl mannitol.

19. A saccharide-based epoxy monomer of claim 11, wherein said epoxy comprises 1,2,3,4,5,6-hexa-O-epoxymethallyl mannitol.

20. A saccharide-based epoxy monomer of claim 11, wherein said epoxy comprises epoxymethallyl sucrose.

21. A saccharide-based epoxy monomer of claim 11, wherein said epoxy comprises 1′,2,3,3′,4,4′,6,6′-octa-O-epoxymethallyl sucrose

22. A saccharide-based epoxy monomer of claim 11, wherein said epoxy comprises epoxymethallyl cellulose.

23. A composition of matter comprising a polymerized epoxy mixture comprising a plurality of saccharide-based epoxy monomers of claim 11 and a curing agent.

24. An epoxy mixture of claim 23, wherein said curing agent is selected from the group consisting of ureas, urethanes, amines, thiols, phenols, amides, ketimines, sulfides, mercaptans, amino acids, imidazoles, amines, diamines, polyamines, diethylenetriamine, triethylenetetramine, tetraethylenepentamine, dicyandiamide and aminoplasts, thiols, polysulfides and polymercaptans, and mixtures thereof.

25. An epoxy mixture of claim 23, wherein said saccharide-based epoxy monomers are epoxymethallyl sucroses, and said curing agent is selected from the group consisting of polyamides, polyamidoamines, diethylenetriamines and mixtures thereof.

26. An epoxy mixture of claim 23, wherein said saccharide-based epoxy monomers are epoxymethallyl xylitols, and said curing agent is selected from the group consisting of polyamides, polyamidoamines, diethylenetriamines and mixtures thereof.

27. An epoxy mixture of claim 23, wherein said saccharide-based epoxy monomers are epoxymethallyl mannitols, and said curing agent is selected from the group consisting of polyamides, polyamidoamines, diethylenetriamines and mixtures thereof.

28. An epoxy mixture of claim 23, wherein said saccharide-based epoxy monomers are epoxymethallyl celluloses, and said curing agent is selected from the group consisting of polyamides, polyamidoamines, diethylenetriamines and mixtures thereof.

29. An epoxy mixture of claim 23, wherein said curing agent is a hardener/catalyst selected from the group consisting of latent acid catalysts, borontriflouride ethylamine complex, aryl iodonium salts, aryl sulfonium salts and aryl selenium compounds, and mixtures thereof.

30. A composition of matter comprising an adhesive comprising an epoxy mixture of claim 23 wherein:
a. said saccharide-based epoxy monomers are selected from the group consisting of epoxyallyl sucrose, epoxycrotyl sucrose, epoxymethallyl sucrose, epoxy methallyl sorbitols and epoxymethallyl xylitols, epoxymethallyl celluloses and mixtures thereof; and
b. said curing agent is selected from the group consisting of aliphatic and aromatic polyamines, polyamides, polyamidoamines, polythiols, polymercaptans and amino acids, and mixtures thereof.

31. An adhesive of claim 30, wherein said saccharide-based epoxy monomers are epoxymethallyl sucroses, and said curing agent is selected from the group consisting of polyamides, polyamidoamines, diethylenetriamines and mixtures thereof.

32. An adhesive of claim 30, wherein said saccharide-based epoxy monomers are epoxymethallyl xylitols, and said curing agent is selected from the group consisting of polyamides, polyamidoamines, diethylenetriamines and mixtures thereof.

33. An adhesive of claim 30, wherein said saccharide-based epoxy monomers are epoxymethallyl mannitols, and said curing agent is selected from the group consisting of polyamides, polyamidoamines, diethylenetriamines and mixtures thereof.

34. An adhesive of claim 30, wherein said saccharide-based epoxy monomers are epoxymethallyl celluloses, and said curing agent is selected from the group consisting of polyamides, polyamidoamines, diethylenetriamines and mixtures thereof.

35. An adhesive of claim 30, wherein said saccharide-based epoxy monomers are epoxymethallyl saccharides, and said curing agent is selected from the group consisting of diethylenetriamines, triethylenetetramines, tetraethylenepentamines and thiols, and mixtures thereof.

36. An adhesive of claim 30, wherein said saccharide-based epoxy monomers are a mixture of epoxyallyl saccharides, and said curing agent is selected from the group consisting of polyamides and polyamidoamines, and mixtures thereof.

37. A composition of matter comprising a coating comprising an epoxy mixture of claim 23 and further comprising a viscosity modifier, wherein:
a. said saccharide-based epoxy monomers are selected from the group consisting of epoxyallyl sucrose, epoxycrotyl sucrose, epoxymethallyl sucrose, epoxymethallyl sorbitols, epoxymethallyl xylitols and epoxymethallyl celluloses, and mixtures thereof;
b. said curing agent is selected from the group consisting of aliphatic and aromatic polyamines, polyamides, polyamidoamines, polythiols, polymercaptans and amino acids, and mixtures thereof; and
c. said viscosity modifier comprises a solvent that dissolves and lowers the viscosity of said curing agent.

38. A coating of claim 37, wherein said viscosity modifier is selected from the group consisting of methylethylketone, 2-pentanone, cyclohexanone, xylene, cresol and ethyleneglycoldimethylether, and mixtures thereof.

39. A coating of claim 37, wherein said saccharide-based epoxy monomers are epoxymethallyl sucrose, said viscosity modifier is methylethylketone, and said curing agent is selected from the group consisting of metaxylenediamine, diethylenetriamine and polyamidoamine, and mixtures thereof.

40. A composition of matter comprising a composite material comprising an epoxy mixture of claim 23 and further comprising a filler.

41. A composite of claim 40, wherein said filler is vegetable matter.

42. A composite of claim 40, wherein said filler is selected from the group consisting of bagasse, kenaf, nonwoven cotton, wheat straw, rice hull, bamboo, defoliated plant matter and sawdust, and mixtures thereof.

43. A composite of claim 40 and further comprising concrete filter, wherein:
a. said saccharide-based epoxy monomers are selected from the group consisting of epoxycrotyl saccharide-based monomers and epoxymethallyl saccharide-based monomers, and mixtures thereof;
b. said curing agent is selected from the group consisting of amines, polyamines, amides, polyamides, thiols, polythiols, polymercaptans, anhydrides, amino acids and polyanhydrides, and mixtures thereof; and
c. said filler is selected from the group consisting of bagasse, kenaf, wheat straw, rice hull, bamboo, defoliated plant matter and sawdust, and mixtures thereof.

44. A composite of claim 43, wherein:
a. said saccharide-based epoxy monomers are 1′,2,3,3′,4,4′,6, 6′-octa-O-epoxymethallyl sucrose;
b. said curing agent is selected from the group consisting of diethylenetriamine and methylene-phylene diamene; and
c. said fillers are selected from the group consisting of bagasse, kenaf and defoliated cotton plant, and mixtures thereof.

45. A composite of claim 43, wherein:
a. said saccharide-based epoxy monomers are selected from the group consisting of 1′,2,3,3′,4,4′,6,6′-octa-O-epoxymethallyl sucrose and 1′,2,3,3′,4,4′,6,6′-octa-O-crotyl sucrose, and mixtures thereof;
b. said curing agent is norbornadiene anhydride in the presence of a catalytic amount of a catalyst selected from the group consisting of trioctylamine and metaxylenediamine, and mixtures thereof; and
c. said fillers are selected from the group consisting of bagasse, kenaf and defoliated cotton plant, and mixtures thereof.

46. A composite of claim 40, wherein:
a. said saccharide-based epoxy monomers are epoxyallyl saccharides;
b. said curing agent is selected from the group consisting of polyamidoamines and polythiols, and mixtures thereof, and
c. said filler is long fiber matter.

47. A composite of claim 46, wherein said filler is selected from the group consisting of nonwoven cotton, nonwoven kenaf, nonwoven jute and fiberglass, and mixtures thereof.

48. A composite of claim 47, further comprising polyester and polypropylene.

49. A method of making an unepoxidized monomer of claim 1, comprising the steps of reacting a saccharide-based monomer in aqueous sodium hydroxide with methallyl chloride.

50. A method of making unepoxidized monomers described in claim 49, comprising the steps of:
a. combining said saccharide-based monomers and aqueous NaOH in a vessel, and heating for about one hour and thirty minutes;
b. cooling same, and adding cold methallyl chloride;
c. equilibrating the internal temperature of same, and stirring;
d. placing said vessel in an ice bath, depressurizing same, and diluting said contents with ice water;
e. extracting organic contents with cold ethyl acetate;
f. washing said extraction, drying same, filtering same, and concentrated same in vacuo.

51. A method of making unepoxidized monomers described in claim 49, comprising the steps of:
a. combining about 0.584 moles of sucrose in about 7.011 moles of aqueous NaOH in sealed pressure vessel, heating to the range of between about 80° C. and 100° C. for about thirty minutes, and maintaining said temperature for about one hour;
b. cooling the contents of said vessel to about 50□C, and adding about 7.011 moles cold methallyl chloride, then pressurizing said vessel with nitrogen gas;
c. equilibrating the internal temperature of said vessel to about 80□C over two hours, and stirring the contents for about overnight;
d. cooling said vessel to about room temperature, placing said vessel in an ice bath, depressurizing said vessel and diluting said contents with ice water;
e. transferring said contents to a separatory funnel with ice water, and extracting an organic layer of same with cold ethyl acetate;
f. washing serially said extraction with water and brine, drying same over sodium sulfate, filtering same, and concentrating same in vacuo overnight.

52. A method of making unepoxidized monomers described in claim 49, comprising the steps of:
a. combining about 0.779 moles of sorbitol in about 7.011 moles of aqueous NaOH in sealed pressure vessel, heating to the range of between about 80° C. and 100° C. for about thirty minutes, and maintaining said temperature for about one hour;
b. cooling the contents of said vessel to about 50□C, and adding about 7.011 moles cold methallyl chloride, then pressurizing said vessel with nitrogen gas;
c. equilibrating the internal temperature of said vessel to about 80□C over two hours, and stirring the contents for about overnight;
d. cooling said vessel to about room temperature, placing said vessel in an ice bath, depressurizing said vessel and diluting said contents with ice water;
e. transferring said contents to a separatory funnel with ice water, and extracting an organic layer of same with cold ethyl acetate;
f. washing serially said extraction with water and brine, drying same over sodium sulfate, filtering same, and concentrating same in vacuo overnight.

53. A method of making an unepoxidized monomers described in claim 46, comprising the steps of:
a. combining about 0.935 moles of xylitol in about 7.011 moles of aqueous NaOH in sealed pressure vessel, heating to the range of between about 80° C. and 100° C. for about thirty minutes, and maintaining said temperature for about one hour;
b. cooling the contents of said vessel to about 50□C, and adding about 7.011 moles cold methallyl chloride, then pressurizing said vessel with nitrogen gas;
c. equilibrating the internal temperature of said vessel to about 80□C over two hours, and stirring the contents for about overnight;
d. cooling said vessel to about room temperature, placing said vessel in an ice bath, depressurizing said vessel and diluting said contents with ice water;
e. transferring said contents to a separatory funnel with ice water, and extracting an organic layer of same with cold ethyl acetate;
f. washing serially said extraction with water and brine, drying same over sodium sulfate, filtering same, and concentrating same in vacuo overnight.

54. A method of making an unepoxidized monomer of claim 1, comprising the steps of reacting a saccharide-based monomer in sodium hydride in dimethylsulfoxide with methallyl chloride.

55. A method of making unepoxidized monomers described in claim 54, comprising the steps of:
a. preparing a solution of sodium hydride (60% in oil, 8.4 g, 210 mmol, 1.8 eq. per OH group of sucrose) washed serially with dry hexanes (4×15 mL) in dimethylsulfoxide;
b. cooling said solution to about 10° C.;
c. adding a solution of sucrose (5.0 g, 14.62 mmol, 116.8 mmol OH groups) in 30 mL dimethylsulfoxide;
d. heating to about 35-40° C. and stirring for about 90 minutes;
e. cooling a resulting mixture to about 10° C. and treating with methallyl chloride (13.6 g, 14.8 mL, 150.22 mmol, 1.3 eq. per OH group, added over 30 minutes), allowing same to attain a temperature of about 40° C., and then stirring overnight;
f. quenching said mixture with 5% aqueous sodium hydroxide (30 mL) at about 15° C., diluted with water (500 mL), and extracting with ethyl acetate (4×100 mL);
g. Combining the resulting organic layers, washing serially with water, hydrogen peroxide (5% solution in water), water, and brine (3×150 mL each), drying over anhydrous sodium sulfate, filtering through charcoal, and then concentrating in vacuo.

56. A method of making unepoxidized monomers described in claim 55, wherein sorbitol (3.56 g, 19.43 mmol) is exchanged for said sucrose (5.0 g, 14.62 mmol,).

57. A method of making unepoxidized monomers described in claim 55, wherein xylitol (3.55 g, 23.33 mmol) is exchanged for said sucrose (5.0 g, 14.62 mmol).

58. A method of making an epoxidized monomer of claim 11, comprising the steps of epoxidizing methallyl saccharide-based monomers with peracids to generate epoxymethallyl saccharides.

59. A method of making epoxidized monomers described in claim 58, comprising the steps of:
a. refrigerating a vessel charged with a methallyl saccharide-based monomer dissolved in ethyl acetate, and adding sodium acetate;
b. cooling said vessel, and adding peracetic acid dropwise;
c. heating said vessel to 10□C, and stirring said contents;
d. diluting said contents with ethyl acetate, and washing serially with cold water, cold aqueous saturated sodium carbonate and brine;
e. separating an organic layer, drying same, filtering same, and concentrating epoxymethallyl saccharide-based epoxy monomers.

60. A method of making epoxidized monomers described claim 58, comprising the steps of:
a. placing a vessel in a refrigeration bath, charging same with methallylsucrose dissolved in ethyl acetate, and adding sodium acetate in an amount equal to about 10% of the number of moles of peracetic acid to be added later;
b. cooling said vessel to about 5° C. and adding about 5.751 moles of peracetic acid dropwise over two hours;
c. heating to about 10° C., and stirring said contents overnight;
d. diluting said contents with ethyl acetate, transferring same to a separatory funnel and washing same serially with cold water, cold aqueous saturated sodium carbonate and brine;
e. separating an organic layer, drying same over anhydrous sodium carbonate, filtering same, and concentrating same in vacuo.

61. A method of making epoxidized monomers described claim 58, wherein said saccharide-based monomer is sorbitol.

62. A method of making epoxidized monomers described claim 58, wherein said saccharide-based monomer is xylitol.

63. A method of making a polymerized epoxy mixture of claim 23, comprising the step of mixing said saccharide-based epoxy monomers and said curing agent.

64. A method of making an adhesive of claim 30, comprising the step of mixing said saccharide-based epoxy monomers and said curing agent.

65. A method of making a coating of claim 36, comprising the step of mixing said saccharide-based epoxy monomers and said curing agent and said viscosity modifier.

66. A method of making a composite of claim 40, comprising the step of mixing said saccharide-based epoxy monomers and said curing agent and said filler.

67. A method of making a composite as described of claim 43, comprising the steps of:
a. mixing said vegetable matter and concrete in water;
b. in a separate container, mixing said epoxy, curing agent and viscosity modifier;
c. mix both mixture a. and mixture b. together;
d. molding same to desired shape;
e. heat cure, first to about 80° C. until stiffening begins, then to about 120° C. degrees until fully cured.

68. A method of making a composite as described of claim 48, comprising the steps of:
a. webbing said filler, polyester and polypropylene;
b. applying said mixture of said saccharide-based epoxy monomers and curing agent; and
c. configuring said webbing mixture to desired shape.
